(19) United States
(12) Patent Application Publication
(10) Pub. No.: US 2006/0188513 A1
(43) Pub. Date: Aug. 24, 2006

(54) Novel inhibin-related multiple antigenic peptide compositions that enhance production performance in avians

(76) Inventors: Gary G. Cadd, Grapevine, TX (US); Daniel G Satterlee, Prairieville, LA (US); William C Fioretti, Addison, TX (US)

Correspondence Address: JOHN S. PRATT, ESQ;KILPATRICK STOCKTON, LLP, 1100 PEACHTREE STREET, ATLANTA, GA, 30309, US

(21) Appl. No.: 10/544,510

(22) PCT Filed: Nov. 7, 2003
(86) PCT No.: PCT/US03/35626

§ 371 Date: Feb. 23, 2006

(30) Foreign Application Priority Data
Nov. 7, 2002 (US) 60424618

(51) Int. Cl. A61K 39/00; A61K 38/22

(52) U.S. Cl. 424/185.1; 514/12

(57) ABSTRACT

The present invention provides novel compositions of matter comprising MAP compositions comprising at least 2 inhibin-related peptides linked to a backbone. These compositions are immunogenic and enhance production performance when administered together with an acceptable carrier to animals, especially avians.

### FIELD OF THE INVENTION

The present invention relates to novel compositions of matter comprising inhibin-related, multiple antigenic peptide compositions that enhance production performance when administered with an acceptable carrier to animals, especially avians.

### BACKGROUND

Avian species provide a significant source of food in terms of meat and eggs. Enhancing production performance of avian species is of significant economic value to an industry currently enjoying high growth due to an ever increasing product demand. To satisfy consumer demand and maintain their competitive edge in meat pricing, broiler and turkey breeders will continue to produce as many hatching eggs as possible. Therefore, any method capable of increasing egg production by even small amounts would generate significant economic benefits.

As an example, the scenario of a single broiler breeder hen (per hen housed basis) laying 15 additional eggs during one production cycle (approximately 1 year in length) is as follows. Such a case would, at current market chick prices ($0.16/chick), result in the generation of an extra $2.00 income from the sale of additional hatchlings and $10-12.00 as proceeds from the added sales of chicken meat generated from the grow-out of these chicks (after considering appropriate deductions for feed costs and fixed costs). Because the estimated broiler breeder hen population is believed to be in excess of 60 million hens in the United States, economic gain would be approximately $750+ million. Including the increased revenue that would be generated by enhancing production performance in turkey breeders (as well as in hens comprising the specialized poultry businesses of ducks, geese and quail, and including the chickens discussed above) $1-2 billion is estimated for the overall economic gain that might be realized from the meat-side of the poultry industry, including all poultry raised for consumption of flesh.

The phrase “enhancing production performance” is understood by those of ordinary skill in the art to denote an increase in one or more of the following in female birds: fertility, accelerated onset of egg lay; accelerated onset of maximum egg production; prolonged persistence of egg lay; increased intensity of egg lay; or increased total lifetime egg lay. The phrase also includes improved feed conversion ratios; improved egg shell quality; or improved resistance to adverse laying conditions such as heat stress, overcrowding, poor nutrition, and noise. The phrase means an increase in one or more of the following in males: fertility, accelerated onset of puberty or production of sperm; accelerated onset of maximum sperm production; increased persistence of sperm production; increased intensity of sperm production (sperm count); increased ejaculate volume; improved sperm viability; increased testosterone production; or increased libido.

Further, the improvement of the production performance of all poultry is needed to increase the amount of poultry produced for consumption and to improve the efficiency of such production, or feed conversion ratio. Accordingly, there remains a need for a composition and method of improving or enhancing production performance for all poultry, including chickens, turkeys, ducks, quail, and geese, among others.

There also is a need for a composition and method of enhancing production performance in ratites such as ostrich, and emu and in exotic birds, such as the Psittaciformes. Psittaciformes include parrots, and are a monofamilial order of birds that exhibit zygodactylism and have a strong hooked bill. A parrot is defined as any member of the avian family Psittacidae (the single family of the Psittaciformes), distinguished by the short, stout, strongly hooked beak.

The need for a composition and method for enhancing production performance is not limited to birds. There remains a need for an effective composition and method for enhancing production performance in many animals. For example, there is a continued need for enhancing production performance in most animals that are raised agriculturally, such as pigs, cows, and sheep. There is also a continued need of enhancing production performance in fur bearing animals such as mink, fox, otter, ferret, raccoons, and in rodents such as rats, mice, gerbils, and hamsters used as pets and as laboratory research subjects, and there is an increased need for other animals whose hides are used for decorative purposes.

A composition and method for enhancing production performance is also needed to increase the population of many animals such as exotic or endangered species to avoid their extinction. There is further a continuing need for enhancing production performance in animals used for racing, entertainment, or showing (competitions) such as horses, dogs, cats, zoo animals, and circus animals. As shown by the increased demands for infertility treatment of humans, there is also a need for enhancing production performance in humans. Accordingly, there remains a need for a composition and method for enhancing production performance in many animals.

### SUMMARY OF THE INVENTION

The present invention relates, in general, to a method of enhancing the production performance of animals, by administering to the animal a composition comprising a multiple antigenic peptide (MAP) composition, comprised of immunogenic peptides linked to a backbone of amino acids, together with an acceptable carrier. An effective amount of the MAP composition is administered to an animal such that an immunological response occurs in the animal against the MAP composition. It is to be understood that the method of the present invention enhances production performance of animals that produce inhibin. Preferably the peptides are inhibin-related peptides. Preferably, the animal is a bird. Preferably the bird is a poultry bird. More preferably, the bird is a chicken, turkey, duck, goose or quail. Another preferred bird is a ratite, such as an emu, an ostrich, a rhea, or a cassowary.

The present invention further relates to novel compositions comprising MAP compositions comprising at least two inhibin-related immunogenic peptides linked to a backbone of amino acids, which are capable of enhancing production performance when administered with an acceptable carrier to animals, especially avians. More particularly, the present invention is directed to compositions comprising MAP compositions comprising immunogenic peptide fragments of the mature alpha subunit of the inhibin protein, linked to a backbone of amino acids. The MAP compositions of the present invention also comprise one or more of the mature alpha subunit of the inhibin protein, linked to a backbone of amino acids. The inhibin protein can be from any species, preferably avian inhibin, mammalian inhibin, piscine inhibin, or reptilian inhibin. The present invention also includes modified inhibin peptides such that individual amino acids may be conservatively substituted with other natural or non-natural amino acids.

The present invention is also directed to a method of enhancing production performance in animals via the administration of the MAP compositions of the present invention which comprise at least two immunogenic peptide fragments of inhibin, or conservative substitutions thereof, linked to a backbone comprising amino acids. In one embodiment, the method comprises administering an effective amount of the MAP composition to a female animal. In another embodiment, the method comprises administering an effective amount of the MAP composition to a male animal. Preferably, an immunological response occurs in the animal directed against the MAP composition. More preferably, the immunological response that occurs in the animal is also directed against the endogenous inhibin protein produced by the animal, or a fragment thereof.

The method of the present invention enhances production performance in female animals that produce inhibin, such as mammals, reptiles, fish, and birds. Preferably, this method enhances production performance in female birds. More preferably, this method enhances production performance in chickens, turkeys, quail, geese, ducks, ostriches, emus, and rhea. Unexpectedly, the method of the present invention accelerates the onset of puberty or egg lay in animals. Also, the method of the present invention unexpectedly accelerates the onset of maximum egg lay in an animal. Further, the method of the present invention increases the intensity of egg lay of an animal. Further still, the method of the present invention surprisingly prolongs the persistence of maximum egg lay in animals. Still further, the method unexpectedly increases the lifetime total egg lay of an animal. The method of the present invention also improves the feed conversion ratio of a bird. The method of the present invention is particularly effective in birds that are lower in body weight than the industry standard. Accordingly, the present invention significantly enhances production performance and particularly egg lay in birds that are not optimally managed with regard to feeding. Also, the method of the present invention unexpectedly reduces or eliminates the effect of adverse laying conditions on egg lay rates of animals exposed to such conditions. Such adverse conditions include elevated temperatures, overcrowding, poor nutrition, lower body weight, and noise. The method of the present invention also increases the libido, and therefore the reproductive potential, of a female bird.

The method of the present invention also improves production performance in male animals that produce inhibin, such as mammals, reptiles, and birds. In a preferred embodiment, the method of the present invention improves production performance in male birds including but not limited to chickens, turkeys, quail, ducks, geese, ostriches, emus, and rhea. More particularly, the method of the present invention increases testosterone levels in male animals. Similarly, the method of the present invention increases the onset of puberty or sperm production in male animals. Also, the method of the present invention accelerates the onset of maximum sperm production in a male animal. Further, the method of the present invention unexpectedly increases the intensity of sperm production (sperm count) by a male animal. Further still, the method of the present invention prolongs the persistence of maximum sperm production in animals. Also, the method improves sperm viability in animals. The method of the present invention also improves the feed conversion ratio of an animal. The method of the present invention is particularly effective in animals that are lower in body weight than the industry standard. Accordingly, the present invention significantly enhances production performance in animals that are not optimally managed with regard to feeding. Still further, the method unexpectedly reduces or eliminates the effect of adverse conditions on sperm production of animals exposed to such conditions. Such adverse conditions include elevated temperatures, overcrowding, poor nutrition, and noise. The method of the present invention also surprisingly increases the libido, and therefore, the reproductive potential, of a male animal particularly birds.

By enhancing development of male and female animals, especially birds, the present invention also facilitates earlier harvesting of animals for any of their products, including their meat that may be consumed. The present invention also decreases the cost of production of animals, particularly birds, for their meat, eggs and other products, by decreasing the feed conversion ratio.

Accordingly, the method of the present invention ameliorates the negative impact on egg lay rates of poultry exposed to adverse egg laying conditions. This aspect of the present invention is significant as poultry are often raised in open, uncontrolled environments. Poultry stocks are often exposed to adverse conditions such as underfeeding, elevated temperatures, and other extreme weather conditions that they are not acclimated to, which thereby decrease egg lay rates in the poultry industry.

As stated above, the method of the present invention is used to enhance production performance of any animal that produces inhibin, including, but not limited to the following: most animals that are raised agriculturally, such as pigs, cows, sheep, turkeys, quail, ducks, geese, chickens, and fish; fur bearing animals such as mink, fox, otter, ferret, rabbits and raccoon; laboratory animals such as rats, mice, gerbils, and guinea pigs; animals whose hides are used for decorative purposes such as alligators and snakes; exotic or endangered species; animals used for racing, entertainment, or showing (competitions) such as horses, dogs, cats, zoo animals, and circus animals; and, humans.

The method of the present invention enhances production performance in other avians including ratites, psittaciformes, falconiformes, piciformes, strigiformes, passeriformes, coraciformes, ralliformes, cuculiformes, columbiformes, galliformes, anseriformes, and herodiones. More particularly, the method of the present invention may be used to enhance production performance of an ostrich, emu, rhea, chicken, turkey, duck, goose, quail, partridge, pheasant, kiwi, cassowary, parrot, parakeet, macaw, falcon, eagle, hawk, pigeon, cockatoo, song birds, jay bird, blackbird, finch, warbler, canary, toucan, mynah, or sparrow.

Accordingly, it is an object of the present invention to provide novel MAP compositions comprising at least two inhibin-related peptides, fragments thereof, or conservative substitutions thereof, covalently bound to a backbone comprising amino acids.

It is another object of the present invention to provide novel MAP compositions comprising the mature alpha subunit of inhibin, or conservative substitutions thereof, covalently bound to a backbone comprising amino acids.

It is another object of the present invention to provide novel MAP compositions comprising the mature alpha subunit of inhibin, fragments thereof, or conservative substitutions thereof, covalently bound to a backbone comprising amino acids.

Yet another object of the present invention is to provide novel MAP compositions comprising conservatively substituted fragments of inhibin covalently bound to a backbone comprising amino acids.

It is another object of the present invention to provide novel MAP compositions comprising peptide fragments of inhibin, covalently bound to a backbone comprising amino acids, which induce an immunological response in an animal upon administration to the animal with an acceptable carrier.

Another object of the present invention is to provide novel MAP compositions comprising peptide fragments of inhibin covalently bound to a backbone comprising amino acids, that induce an immunological response in a bird upon administration to the bird with an acceptable carrier.

Yet another object of the present invention is to provide novel MAP compositions comprising peptide fragments of inhibin covalently bound to a backbone, comprising amino acids that induce an immunological response in a female bird upon administration to the female bird with an acceptable carrier.

Still another object of the present invention is to provide novel MAP compositions comprising peptide fragments of inhibin covalently bound to a backbone, comprising amino acids that induce an immunological response in a male bird upon administration to the male bird with an acceptable carrier.

An object of the present invention is to provide novel MAP compositions comprising peptide fragments of inhibin covalently bound to a backbone, comprising amino acids that increase production performance in a bird upon administration to the bird with an acceptable carrier.

Another object of the present invention is to provide novel MAP compositions comprising peptide fragments of inhibin covalently bound to a backbone, comprising amino acids that increase production performance in a female bird upon administration to the female bird with an acceptable carrier.

Still another object of the present invention is to provide novel MAP compositions comprising peptide fragments of inhibin covalently bound to a backbone, comprising amino acids that increase production performance in a male bird upon administration to the male bird with an acceptable carrier.

Yet another object of the present invention is to provide novel MAP compositions comprising peptide fragments of inhibin, covalently bound to a backbone, comprising amino acids, that increase production performance, wherein production performance is evidenced by enhanced fertility, acceleration in the onset of puberty, increase in the onset of egg lay, increase in the duration of egg lay, increase in the intensity of egg lay in a female bird, decrease in feed conversion ratio, or decrease in the time before harvesting the chicks of injected birds for meat, after administration to the female bird with an acceptable carrier.

Another object of the present invention is to provide novel MAP compositions comprising peptide fragments of inhibin covalently bound to an amino acid backbone, which increase production performance in a male bird after administration to the male bird with an acceptable carrier, wherein production performance is evidenced by enhanced fertility, accelerated onset of puberty or production of sperm; accelerated onset of maximum sperm production; increased persistence of sperm production; increased intensity of sperm production (sperm count); increased ejaculate volume; improved sperm motility and viability; increased testosterone production; enhanced fertility, increased libido or a decrease in the time before harvesting the chicks for meat of females mated with injected males.

An advantage of the present invention is to reduce or eliminate the industry practice of supplementing aged breeder flocks of birds with younger males to maintain optimum fertility.

It is an object of the present invention to provide a method for enhancing production performance in poultry.

It is another object of the present invention to provide a method for enhancing production performance in chickens.

Yet another object of the present invention is to provide a method for enhancing production performance in turkeys.

It is an object of the present invention to provide a method for enhancing production performance in quail.

Another object of the present invention is to provide a method for enhancing production performance in ducks.

Yet another object of the present invention is to a method for enhancing production performance in geese.

It is another object of the present invention to provide a method for enhancing production performance in reptiles.

Yet another object of the present invention is to provide a method for enhancing production performance in mammals.

Still another object of the present invention is to provide a method for enhancing production performance in fish,

It is further an object of the present invention to provide a method for enhancing production performance in humans.

These and other objects, features and advantages of the present invention will become apparent after a review of the following detailed description of the disclosed embodiments and the appended claims.

### BRIEF DESCRIPTION OF THE DRAWINGS

FIG. 1 is a schematic representation of the average age at first egg lay (FIRST) (open histogram bars) and 25% egg production (T-FIVE)(shaded histogram bars) in birds injected with ¹⁻²⁶INH-MAP at dosages of 0.05, 0.1 or 0.2 mg/bird. Bars with different letters (a, b) are statistically different (P<0. 10).

### DETAILED DESCRIPTION

The present invention relates, in general, to a method of enhancing the production performance of animals, by administering to the animal a MAP composition comprised of at least two immunogenic fragments of inhibin protein, linked to a backbone comprising amino acids, in an acceptable carrier. An effective amount of the MAP composition is administered to an animal such that an immunological response occurs in the animal against the MAP composition. It is to be understood that the method of the present invention enhances production performance of animals that produce inhibin. Preferably, the animal is a bird. More preferably, the bird is a chicken, turkey, quail, goose or duck. Still other preferred birds are pheasants and partridge. Yet another preferred bird is a ratite, such as, an emu, an ostrich, a rhea, or a cassowary. The present invention further relates to novel MAP compositions described herein.

After the following definitions, the composition of the present invention is described in detail, followed by a detailed description of the methods of the present invention.

Definitions

The term “bird” or “fowl,” as used herein, is defined as a member of the Aves class of animals which are characterized as warm-blooded, egg-laying vertebrates primarily adapted for flying. Avians include without limitation ratites, psittaciformes, falconiformes, piciformes, strigiformes, passeriformes, coraciformes, ralliformes, cuculiformes, columbiformes, galliformes, anseriformes, and herodiones. The term “Ratite,” as used herein, is defined as a group of flightless, mostly large, running birds comprising several orders and including the emus, ostriches, kiwis, and cassowaries. The term “Psittacifornes”, as used here, include parrots, and are a monofamilial order of birds that exhibit zygodactylism and have a strong hooked bill. A “parrot” is defined as any member of the avian family Psittacidae (the single family of the Psittaciformes), distinguished by the short, stout, strongly hooked beak. The term “chicken” as used herein denotes chickens used for table egg production, such as egg-type chickens, chickens reared for public meat consumption, or broilers, and chickens reared for both egg and meat production (“dual-purpose” chickens). The term “chicken” also denotes chickens produced by primary breeder companies, or chickens that are the parents, grandparents, great-grandparents. etc. of those chickens reared for public table egg, meat, or table egg and meat consumption.

The term “egg” is defined herein as a large female sex cell enclosed in a porous, calcarous or leathery shell, produced by birds and reptiles. “Egg production by a bird or reptile”, as used herein, is the act of a bird or reptile laying an egg, or “oviposition”. The term “ovum” is defined as a female gamete, and is also known as an egg. Therefore, egg production in all animals other than birds and reptiles, as used herein, is defined as the production and discharge of an ovum from an ovary, or “ovulation”. Accordingly, it is to be understood that the term “egg” as used herein is defined as a large female sex cell enclosed in a porous, calcarous or leathery shell, when a bird or reptile produces it, or it is an ovum when it is produced by all other animals.

The terms “onset of egg lay”, “first egg lay” and “puberty”, in reference to birds are used interchangeably herein, and denote when a bird lays its first egg. Accordingly, “accelerating the onset” of egg lay or puberty in avians, as used herein, denotes inducing an earlier date of first egg lay than a bird would normally have. Similarly, “puberty” and “onset of sperm production” in males are used interchangeably. The measurement of pubic spread (e.g., 3 fingers or greater) is considered to be a marker of sexual development that predicts that the “onset (day) of puberty (first egg)” is about to, or may have already, happened. Likewise, T-FIVE is an arbitrary marker or predictor of puberty that says a flock did, on average, reach 25% egg production on a given day. The FIRST (age at first oviposition) measurement clearly defines the exact date of “puberty”.

The phrases “enhancing production performance”, “improving production performance” and “increasing production performance” are used interchangeably to denote an improvement in one or more of the following areas: enhanced fertility; accelerated onset of puberty (egg lay or ovulation in females; sperm production in males); accelerated onset of maximum egg lay or ovulation in females or accelerated onset of maximum sperm production in males; increased intensity of production of eggs in females, or of sperm in males; prolonged persistence of egg lay in females or of sperm production in males; increased total lifetime egg lay or ovulation in females; improved feed conversion ratios; improved egg shell quality; improved resistance to adverse conditions such as elevated ambient temperatures, overcrowding, poor or sub-optimal nutrition, and noise; improved sperm viability in males; increased testosterone production in males; increased ejaculate volume; increased libido in males and females; and a decrease in the age at which an animal or its offspring may be harvested for meat or other animal products.

The phrase “intensity of egg lay” is known to those of ordinary skill in the art to denote frequency of egg lay.

The phrase “lifetime total egg lay” of a bird is defined as the total number of eggs laid by a bird during its entire life span. The phrase “hen day egg production” or “HDEP”, as used herein, is defined as the average number of eggs laid by a particular group of hens per day.

The phrase “accelerated onset of maximum egg lay” or “accelerated onset of maximum egg production” as used herein, denotes that the period of time from hatching to when the animal lays eggs or ovulates at 25%, 50%, 75% or any other arbitrarily selected milestone shy of its peak lay rate or ovulation rate, is shorter than the normal period of time from hatching to maximum egg lay.

MAP Compositions of the Present Invention

One embodiment of the present invention is a multivalent ligand that has biological activity to affect production performance and is represented by structural formula I:
B-(L-P)n I
Wherein B is a multilinker backbone, n is an integer from 2 to about 20, each L is a covalent bond or a linking group which may be present or absent, and each P is a peptide having from about 4 to about 115 amino acid residues. At least two of the peptides contain peptide fragments of a production performance homology region (PPR) of inhibin, a hybrid peptide, a peptide derivative of a hybrid peptide or a conservatively substituted peptide. A PPR is a peptide region of any inhibin which may cause an immunological response when administered as a peptide component of a MAP composition of the present invention. These immunological responses against the PPR, or a fragment or conservative substitution thereof, may result in an increase in production performance of the animal receiving the MAP composition containing the PPR, the fragment thereof or the conservative substitution thereof. Each P and each linker or covalent bond are independently chosen and may be the same or different.

Another embodiment of the present invention is a polypeptide multivalent ligand having biological activity to affect production performance. A “polypeptide multivalent ligand” is a repetitive polypeptide chain in which two or more peptides, designated as P, are each separated by a multilinker backbone or peptide spacer, designated as B. A polypeptide multivalent ligand is represented by structural formulae (II and III):
P—(B—P)m-B—P II
wherein m is an integer from zero to about twenty.
Pa—(B)n-Pa III
wherein n is an integer from I to 20, preferably 2 to 10, more preferably 3 to 7, further wherein a is 1 or 2. In a preferred embodiment, a is 2 and n is 3. In this embodiment, two Ps are linked to each of the first and third B in the composition.

The MAP compositions of the present invention may occur in different configurations including but not limited to linear, cyclic and branched configurations. Non-limiting examples of such configurations include the following:

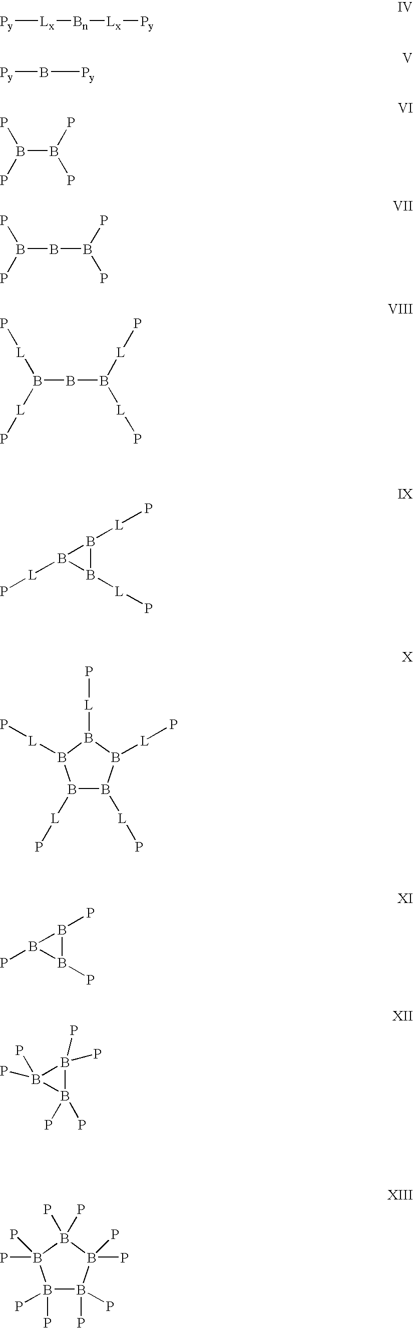

In the preceding structural formulae of exemplary embodiments of MAP compositions, each P is an inhibin-related peptide having from about 4 to about 115 amino acid residues, y is 1 or 2, x is an integer from 1 to 3, and n is an integer from 1 to 20, preferably 2 to 10, more preferably 3 to 7. At least two of the peptides contain peptide fragments or derivatives of a PPR of inhibin, a hybrid peptide or a peptide derivative of a hybrid peptide. Preferably the peptides contain the mature inhibin alpha subunit, peptide fragments or derivatives of a PPR of the mature inhibin alpha subunit, a hybrid peptide or a peptide derivative of a hybrid peptide. Such hybrid peptides or derivatives thereof are preferably substantially homologous to the mature inhibin alpha subunit.

Each B is a backbone structure comprised of at least 2 amino acids and optionally one or more hydrocarbons having from about 2 to about 30 carbons. B is preferably comprised of at least 2 amino acids capable of binding to P. B may optionally include other amino acids that are not capable of binding to P, or hydrocarbon chains (CH₂)ₙ, wherein n is from 1 to 20. Such hydrocarbon chains are preferably saturated. Each peptide P and each B are independently chosen and may be the same or different.

“Substantial homology” exists between two amino acid sequences in a peptide or protein when a sufficient number of amino acid residues at corresponding positions of each amino acid sequence are either identical or structurally related such that a peptide having the first amino acid sequence and a peptide having the second amino acid sequence exhibit similar biological activities. A protein has a PPR when the amino acid sequence of said protein has a subsequence that is substantially homologous to the amino acid sequence of a PPR of inhibin such that a peptide having an amino acid sequence corresponding to said subsequence (or said consensus sequence) modulates production performance when administered to an animal. Generally, there is substantial homology among the amino acid sequences of two PPRs when at least 30%, and preferably at least 40% of the amino acids in one PPR are identical to or structurally related to the amino acid residues in the other PPR. Substantial homology exists between the amino acid sequence of a peptide P and the amino acid sequence of a PPR when a sufficient number of amino acids at corresponding positions in the amino acid sequence of the peptide and PPR (or consensus sequence) are identical or structurally related such that the peptide is capable of affecting production performance. Generally, there is substantial homology between a peptide and a PPR when at least 40%, preferably at least 50% of the amino acids in the peptide are identical to or structurally related to the amino acid residues in the corresponding positions in the PPR, or a subsequence thereof with the ability to affect production performance. “Structurally related” is defined herein below.

One embodiment of the present invention is a peptide derivative of a PPR of any inhibin protein. The peptide derivative has biological activity to affect production performance when administered to an animal. Examples include peptide derivatives of a peptide represented by SEQ ID NOs.: 1 to 30.

A “peptide derivative of a PPR” includes a peptide fragment of a PPR wherein the peptide has an amino acid sequence of the PPR. A “peptide derivative of a PPR” also includes a peptide having a sequence corresponding to a fragment of the PPR. A “PPR fragment” is defined to be a peptide whose amino acid sequence corresponds to a subsequence of a PPR, referred to as a “subsequence”. A subsequence is a sequence of contiguous amino acid residues found within a larger sequence.

A “peptide derivative” also includes a peptide having a “modified sequence” in which one or more amino acid residues in the original sequence or subsequence have been substituted with a naturally occurring amino acid residue or amino acid residue analog (also referred to as a “modified amino acid residue”) or non-natural amino acid residue and an amino acid analog. Suitable peptide derivatives have modified sequences that are substantially homologous to the amino acid sequence of a PPR or to a subsequence of a PPR. Suitable peptide derivatives also include peptides that are substantially homologous to the consensus sequence of the PPRs of inhibin proteins.

In one embodiment of the present invention, a peptide derivative has an amino acid sequence corresponding to a subsequence of a PPR with between about 4 and about 15 amino acid residues. Zero, one, two or three amino acid residues in the peptide derivative can differ from the amino acid residue(s) in the corresponding position of the subsequence of the PPR. For example, if the subsequence is [AA₁]-[AA₂]-[AA₁]-[AA₄]-[AA₅]-[AA₆]-[AA₇]-[AA₈]-[AA₉]-[AA₁₀] and one amino acid residue in the sequence of the peptide derivative differs from the amino acid residue in the corresponding position of the subsequence, then the peptide derivative can be [AA₁′]-[AA₂]-[AA₃]-[AA₄]-[AA₅]-[AA₆]-[AA₇]-[AA₈]-[AA₉]-[AA₁₀], [AA₁]-[AA₂′]-[AA₃]-[AA₄]-[AA₅]-[AA₆]-[AA₇]-[AA₈]-[AA₉]-[AA₁₀], [AA₁]-[AA₂]-[AA₃′]-[AA₄]-[AA₅]-[AA₆]-[AA₇]-[AA₈]-[AA₉]-[AA₁₀], [AA₁]-[AA₂]-[AA₃]-[AA₄′]-[AA₅]-[AA₆]-[AA₇]-[AA₈]-[AA₉]-[AA₁₀], [AA₁]-[AA₂]-[AA₃]-[AA₄]-[AA₅′]-[AA₆]-[AA₇]-[AA₈]-[AA₉]-[AA₁₀], [AA₁]-[AA₂]-[AA₃]-[AA₄]-[AA₅]-[AA₆′]-[AA₇]-[AA₈]-[AA₉]-[AA₁₀], [AA₁]-[AA₂]-[AA₃]-[AA₄]-[AA₅]-[AA₆]-[AA₇′]-[AA₈]-[AA₉]-[AA₁₀], [AA₁]-[AA₂]-[AA₃]-[AA₄]-[AA₅]-[AA₆]-[AA₇]-[AA₈′]-[AA₉]-[AA₁₀], [AA₁]-[AA₂]-[AA₃]-[AA₄]-[AA₅]-[AA₆]-[AA₇]-[AA₈]-[AA₉′]-[AA₁₀], or [AA₁]-[AA₂]-[AA₃]-[AA₄]-[AA₅]-[AA₆]-[AA₇]-[AA₈]-[AA₉]-[AA₁₀′], wherein [AA′] is a naturally occurring or non-naturally occurring or modified amino acid residue different from [AA].

In another aspect of the present invention, a peptide derivative has an amino acid sequence corresponding to an amino acid sequence or to a subsequence of a PPR with between about 4 and about 28 amino acid residues. In yet another embodiment the peptide derivative has an amino acid sequence corresponding to an amino acid sequence or to a subsequence of a PPR with between about 4 and 115 amino acid residues. Zero, one, two, three or four amino acid residues in the peptide derivative can differ from the amino acid residue(s) in the corresponding position of the sequence or subsequence of the PPR.

An “amino acid residue” is a moiety found within a peptide and is represented by —NH—CHR—CO—, wherein R is the side chain of a naturally occurring amino acid. When referring to a moiety found within a peptide, the terms “amino acid residue” and “amino acid” are used interchangeably in this application. An “amino acid residue analog” includes D or L configurations having the following formula: —NH—CHR—CO—, wherein R is an aliphatic group, a substituted aliphatic group, a benzyl group, a substituted benzyl group, an aromatic group or a substituted aromatic group and wherein R does not correspond to the side chain of a naturally-occurring amino acid.

Suitable substitutions for amino acid residues in the sequence of a PPR or a subsequence of a PPR include conservative substitutions that result in peptide derivatives that can stimulate production performance when administered to animals, preferably avians. A conservative substitution is a substitution in which the substituting amino acid (naturally occurring or modified) is structurally related to the amino acid being substituted, i.e., has about the same size and chemical properties as the amino acid being substituted. Thus, the substituting amino acid would have the same or a similar functional group in the side chain as the original amino acid.

A “conservative substitution” also refers to utilizing a substituting amino acid which is identical to the amino acid being substituted except that a functional group in the side chain is protected with a suitable protecting group. Suitable protecting groups are described in Green and Wuts, “Protecting Groups in Organic Synthesis”, John Wiley and Sons, Chapters 5 and 7, 1991, the teachings of which are incorporated herein by reference. Preferred protecting groups are those which facilitate transport of the peptide through membranes, for example, by reducing the hydrophilicity and increasing the lipophilicity of the peptide, and which can be cleaved, either by hydrolysis or enzymatically (Ditter et al., J. Pharm. Sci. 57:783 (1968); Ditter et al., J. Pharm. Sci. 57:828 (1968); Ditter et al., J. Pharm. Sci. 58:557 (1969); King et al., Biochemistry 26:2294 (1987); Lindberg et al., Drug Metabolism and Disposition 17:311 (1989); Tunek et al., Biochem. Pharm. 37:3867 (1988), Anderson et al., Arch. Biochem. Biophys. 239:538 (1985) and Singhal et al., FASEB J. 1:220 (1987)). Suitable hydroxyl protecting groups include ester, carbonate and carbamate protecting groups. Suitable amine protecting groups include acyl groups and alkoxy or aryloxy carbonyl groups, as described above for N-terminal protecting groups. Suitable carboxylic acid protecting groups include aliphatic, benzyl and aryl esters, as described below for C-terminal protecting groups. In one embodiment, the carboxylic acid group in the side chain of one or more glutamic acid or aspartic acid residues in a peptide of the present invention is protected, preferably as a methyl, ethyl, benzyl or substituted benzyl ester, more preferably as a benzyl ester.

Provided below are groups of naturally occurring and modified amino acids in which each amino acid in a group has similar chemical and steric properties. Thus, a conservative substitution can be made by substituting an amino acid with another amino acid from the same group. It is to be understood that these groups are non-limiting, i.e. that there are additional modified amino acids which could be included in each group.
Group I includes leucine, isoleucine, valine, methionine and modified amino acids having the following side chains: ethyl, n-propyl n-butyl. Preferably, Group I includes leucine, isoleucine, valine and methionine.
Group II includes glycine, alanine, valine and a modified amino acid having an ethyl side chain. Preferably, Group II includes glycine and alanine.
Group III includes phenylalanine, phenylglycine, tyrosine, tryptophan, cyclohexylmethyl glycine, and modified amino residues having substituted benzyl or phenyl side chains. Preferred substituents include one or more of the following: halogen, methyl, ethyl, nitro, —NH₂, methoxy, ethoxy and —CN. Preferably, Group III includes phenylalanine, tyrosine and tryptophan.
Group IV includes glutamic acid, aspartic acid, a substituted or unsubstituted aliphatic, aromatic or benzylic ester of glutamic or aspartic acid (e.g., methyl, ethyl, n-propyl iso-propyl, cyclohexyl, benzyl or substituted benzyl), glutamine, asparagine, —CO—NH— alkylated glutamine or asparagines (e.g., methyl, ethyl, n-propyl and iso-propyl) and modified amino acids having the side chain —(CH₂)₃—COOH, an ester thereof (substituted or unsubstituted aliphatic, aromatic or benzylic ester), an amide thereof and a substituted or unsubstituted N-alkylated amide thereof. Preferably, Group IV includes glutamic acid, aspartic acid, methyl aspartate, ethyl aspartate, benzyl aspartate and methyl glutamate, ethyl glutamate and benzyl glutamate, glutamine and asparagine.
Group V includes histidine, lysine, omithine, arginine, N-nitroarginine, β-cycloarginine, thydroxyarginine, N-amidinocitruline and 2-amino-4-guanidinobutanoic acid, homologs of lysine, homologs of arginine and homologs of ornithine. Preferably, Group V includes histidine, lysine, arginine and omithine. A homologue of an amino acid includes from 1 to about 3 additional or subtracted methylene units in the side chain.
Group VI includes serine, threonine, cysteine and modified amino acids having C₁-C₅ straight or branched alkyl side chains substituted with —OH or —SH, for example, —CH₂CH₂OH, —CH₂CH₂CH₂OH or —CH₂CH₂OHCH₃. Preferably, Group VI includes serine, cysteine or threonine.

In another aspect, suitable substitutions for amino acid residues in the sequence of a PPR or a subsequence of a PPR include “severe” substitutions that result in peptide derivatives that affect production performance. Severe substitutions which result in peptide derivatives that affect production performance are much more likely to be possible in positions which are not highly conserved in the PPRs of inhibin proteins than at positions which are highly conserved.

A “severe substitution” is a substitution in which the substituting amino acid (naturally occurring or modified) has significantly different size and/or chemical properties compared with the amino acid being substituted. Thus, the side chain of the substituting amino acid can be significantly larger (or smaller) than the side chain of the amino acid being substituted and/or can have functional groups with significantly different chemical properties than the amino acid being substituted. Examples of severe substitutions of this type include the substitution of phenylalanine or cycohexylmethyl glycine for alanine, isoleucine for glycine, a D amino acid for the corresponding L amino acid or —NH—CH[(—CH₂)₅—COOH]—CO— for aspartic acid. Alternatively, a functional group may be added to the side chain, deleted from the side chain or exchanged with another functional group. Examples of severe substitutions of this type include adding of valine, leucine or isoleucine, exchanging the carboxylic acid in the side chain of aspartic acid or glutamic acid with an amine, or deleting the amine group in the side chain of lysine or omithine. In yet another alternative, the side chain of the substituting amino acid can have significantly different steric and chemical properties than the functional group of the amino acid being substituted. Examples of such modifications include tryptophan for glycine, lysine for aspartic acid and —(CH₂)₄COOH for the side chain of serine. These examples are not meant to be limiting.

A “hybrid peptide”, as used herein, is an inhibin fragment peptide having from about 4 to about 115 amino acid residues. Each amino acid residue (naturally occurring or modified) in the amino acid sequence of a hybrid peptide is: (1) identical to the amino acid residue at the corresponding position in the PPR of the original inhibin protein; (2) an amino acid residue structurally related thereto; (3) identical to the amino acid residue at the corresponding position in the PPR of a second inhibin protein; or, (4) an amino acid residue structurally related thereto. As noted above, replacing an amino acid residue with a structurally related amino acid residue is referred to as a “conservative substitution”.

It is to be understood that the peptide P may be an antigenic peptide from any location in an inhibin molecule from any species. In one preferred embodiment, the peptides are derived from the alpha subunit of inhibin, preferably the mature alpha subunit of inhibin. In another preferred embodiment, the inhibin or the alpha subunit of inhibin is avian inhibin or the alpha subunit of avian inhibin. A preferred embodiment of the present invention is a peptide (P) having a sequence of amino acids AA₁ through AA₂₆ indicated by SEQ ID NO: 1. This sequence is the N terminal 26 amino acids of the mature chicken alpha subunit of inhibin.

SEQ ID NO: 1 Ser Ala Val Pro Trp Ser Pro Ala Ala Leu Ser Leu Leu Gln Arg Pro Ser Glu Asp Val Ala Ala His Thr Asn Cys

In one embodiment of the present invention, four such Ps, each comprised of SEQ ID NO: 1 are linked to an amino acid backbone in a pattern as shown in formula VII. 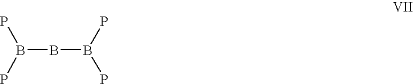
In a preferred embodiment, the amino acid backbone is Bn, wherein n is 3 and each B is Lys. In this embodiment two Ps are linked to Lys₁ and Lys₃ thereby providing the structure shown in structural formula XIV, below which is also called ¹⁻²⁶INH-MAP in the present application. In this structure, no linking structures (L) are included, all the Ps are identical and each is represented by SEQ ID NO: 1. 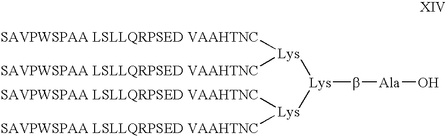
In another preferred embodiment (formula XV), B-Ala-OH is not present on the central lysine in the amino acid backbone. This structure is also called ¹⁻²⁶ INH-MAP in the present application.

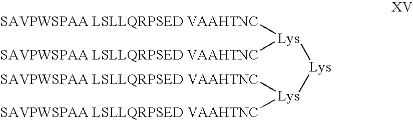

In another embodiment of the present invention, the different Ps represented in a structure, for example structure VII, may be different and may be any number of combinations of SEQ ID NOs: 1 to 30 (below). For example, each P in structures I to XIII could be any one of SEQ ID NOs: 1-30, or combinations thereof.

In another embodiment, when P is SEQ ID NO:1, the individual amino acids may be substituted according in the following manner:
AA₁ is serine, glycine, alanine, cysteine or threonine;
AA₂ is alanine, threonine, glycine, cysteine or serine;
AA₃ is valine, arginine, leucine, isoleucine, methionine, omithine, lysine, N-nitroarginine, β-cycloarginine, γ-hydroxyarginine, N-amidinocitruline or 2-amino-4-guanidinobutanoic acid;
AA₄ is proline, leucine, valine, isoleucine or methionine;
AA₅ is tryptophan, alanine, phenylalanine, tyrosine or glycine;
AA₆ is serine, glycine, alanine, cysteine or threonine;
AA₇ is proline, leucine, valine, isoleucine or methionine;
AA₈ is alanine, threonine, glycine, cysteine or serine;
AA₉ is alanine, threonine, glycine, cysteine or serine;
AA₁₀ is leucine, isoleucine, methionine or valine;
AA₁₁ is serine, glycine, alanine, cysteine or threonine;
AA₁₂ is leucine, isoleucine, methionine or valine;
AA₁₃ is leucine, isoleucine, methionine or valine;
AA₁₄ is glutamine, glutamic acid, aspartic acid, asparagine, or a substituted or unsubstituted aliphatic or aryl ester of glutamic acid or aspartic acid;
AA₁₅ is arginine, N-nitroarginine, β-cycloarginine, γ-hydroxy-arginine, N-amidinocitruline or 2-amino-4-guanidino-butanoic acid
AA₁₆ is proline, leucine, valine, isoleucine or methionine;
AA₁₇ is serine, glycine, alanine, cysteine or threonine;
AA₁₈ is glutamic acid, aspartic acid, asparagine, glutamine or a substituted or unsubstituted aliphatic or aryl ester of glutamic acid or aspartic acid;
AA₁₉ is aspartic acid, asparagine, glutamic acid, glutamine, leucine, valine, isoleucine, methionine or a substituted or unsubstituted aliphatic or aryl ester of glutamic acid or aspartic acid;
AA₂₀ is valine, arginine, leucine, isoleucine, methionine, omithine, lysine, N-nitroarginine, β-cycloarginine, γ-hydroxyarginine, N-amidinocitruline or 2-amino-4-guanidinobutanoic acid;
AA₂₁ is alanine, threonine, glycine, cysteine or serine;
AA₂₂ is alanine, threonine, glycine, cysteine or serine;
AA₂₃ is histidine, serine, threonine, cysteine, lysine or omithine;
AA₂₄ is threonine, aspartic acid, serine, glutamic acid or a substituted or unsubstituted aliphatic or aryl ester of glutamic acid or aspartic acid;
AA₂₅ is asparagine, aspartic acid, glutamic acid, glutamine, leucine, valine, isoleucine, methionine or a substituted or unsubstituted aliphatic or aryl ester of glutamic acid or aspartic acid; and
AA₂₆ is cysteine, histidine, serine, threonine, lysine or omithine.

It is to be understood that the preceding 26 examples of substitutions of amino acids are not limited to SEQ ID NO: 1, and may be applied to any fragment of inhibin, preferably the alpha subunit, or a fragment of the alpha subunit, used a P in the MAP compositions of the present invention, whenever any one of these amino acids is present, provided the MAP composition is effective to enhance production performance when administered to an animal.

Other peptides that may be used as P in the MAP compositions of the present invention include, but are not limited to, SEQ ID NOs. 1 to 30 that follow. It is to be understood that the Ps in a given MAP composition may be the same or different.

In another embodiment, the four Ps in structure VII or in any structure (formulae I to XII) with 2 or more Ps may be the same or different and may be selected from the following:

SEQ ID NO:1

Ser Ala Val Pro Trp Ser Pro Ala Ala Leu Ser Leu
Leu Gln Arg Pro Ser Glu Asp Val Ala Ala His Thr
Asn Cys

SEQ ID NO:2

Ser Ala Val Pro Trp Ser Pro Ala Ala Leu Ser Leu
Leu

SEQ ID NO:3

Ser Ala Val Pro Trp Ser Pro Ala Ala Leu Ser Leu
Leu Gln Arg Pro Ser Glu Asp Val

In another embodiment, the four Ps in structure VII or in any structure (I to XIII) with 2 or more Ps may be the same or different and may be selected from the following:

SEQ ID NO:4

Ala Val Pro Trp Ser Pro Ala Ala Leu Ser Leu Leu
Gln Arg Pro Ser Glu Asp Val Ala Ala His Thr Asn
Cys

SEQ ID NO:5

Pro Trp Ser Pro Ala Ala Leu Ser Leu Leu

SEQ ID NO:6

Ala Val Pro Trp Ser Pro Ala Ala Leu Ser Leu Leu
Gln Arg Pro Ser Glu Asp Val

SEQ ID NO:7

Trp Ser Pro Ala Ala Leu Ser Leu Leu Gln Arg Pro
Ser Glu Asp Val Ala Ala His Thr Asn Cys

In another embodiment, the four Ps in structure VII or in any structure (I to XIII) with 2 or more Ps may be the same or different and may be selected from the following:

SEQ ID NO:8

Leu Gln Arg Pro Ser

SEQ ID NO:9

Asn Cys Arg Arg Ala Ser Leu Asn Leu Ser Phe

SEQ ID NO:10

His Gly Asn Cys Ala Glu Gly His Gly Leu Ser

SEQ ID NO:11

Pro Gly Thr Met Arg Ser

SEQ ID NO:12

Thr Thr Ser Asp Gly Gly Tyr Ser Phe Lys

SEQ ID NO:13

Asp Val Ala Ala His Thr Asn Cys Arg Arg Ala Ser
Leu Asn Leu Ser Phe Glu Glu Leu Gly Trp Asp Asn
Trp Ile Val His Pro Ser Ser Phe Val Phe His Tyr
Cys His Gly Asn Cys Ala Glu Gly His Gly Leu Ser
His Arg Leu Gly Val Gln Leu Cys Cys Ala Ala Leu
Pro Gly Thr Met Arg Ser Leu Arg Val Arg Thr Thr
Ser Asp Gly Gly Tyr Ser Phe Lys Tyr Glu Thr Val
Pro Asn Ile Leu Ala Gln Asp Gys Thr Cys Val

SEQ ID NO:14

Phe Glu Glu Leu Gly Trp Asp Asn Trp Ile Val His
Pro Ser Ser Phe Val Phe His Tyr Cys His Gly Asn
Cys Ala Glu Gly His Gly Leu Ser His Arg Leu Gly
Val Gln Leu Cys Gys Ala Ala Leu Pro Gly Thr Met
Arg Ser Leu Arg Val Arg Thr Thr Ser Asp Gly Gly
Tyr Ser Phe Lys Tyr Glu Thr Val Pro Asn Ile Leu
Ala Gln Asp Cys Thr Cys Val

SEQ ID NO:15

Phe Val Phe His Tyr Cys His Gly Asn Cys Ala Glu
Gly His Gly Leu Ser His Arg Leu Gly Val Gln Leu
Cys Cys Ala Ala Leu Pro Gly Thr Met Arg Ser Leu
Arg Val Arg Thr Thr Ser Asp Gly Gly Tyr Ser Phe
Lys Tyr Glu Thr Val Pro Asn Ile Leu Ala Gln Asp
Cys Thr Cys Val

SEQ ID NO:16

Cys Ala Ala Leu Pro Gly Thr Met Arg Ser Leu Arg
Val Arg Thr Thr Ser Asp Gly Gly Tyr Ser Phe Lys
Tyr Glu Thr Val Pro Asn Ile Leu Ala Gln Asp Cys
Thr Cys Val

SEQ ID NO:17

Val Arg Thr Thr Ser Asp Gly Gly Tyr Ser Phe Lys
Tyr Glu Thr Val Pro Asn Ile Leu Ala Gln Asp Cys
Thr Cys Val

SEQ ID NO:18

Ser Ala Val Pro Trp Ser Pro Ala Ala Leu Ser Leu
Leu Gln Arg Pro Ser Glu Asp Val Ala Ala His Thr
Asn Cys Arg Arg Ala Ser Leu Asn Ile Ser Phe Glu
Glu Leu Gly Trp Asp Asn Trp Ile Val His Pro Ser
Ser Phe Val Phe His Tyr Cys His Gly Asn Cys Ala
Glu Gly His Gly Leu Ser His Arg Leu Gly Val Gln
Leu Cys Cys Ala Ala Leu Pro Gly Thr Met Arg Ser
Leu Arg Val

SEQ ID NO:19

Ser Ala Val Pro Trp Ser Pro Ala Ala Leu Ser Leu
Leu Gln Arg Pro Ser Glu Asp Val Ala Ala His Thr
Asn Cys Arg Arg Ala Ser Leu Asn Ile Ser Phe Glu
Glu Leu Gly Trp Asp Asn Trp Ile Val His Pro Ser
Ser Phe Val Phe His Tyr Cys His Gly Asn Cys Ala
Glu Gly His Gly Leu Ser His Arg Leu Gly Val Gln
Leu Cys Cys

SEQ ID NO:20

Ser Ala Val Pro Trp Ser Pro Ala Ala Leu Ser Leu
Leu Gln Arg Pro Ser Glu Asp Val Ala Ala His Thr
Asn Cys Arg Arg Ala Ser Leu Asn Ile Ser Phe Glu
Glu Leu Gly Trp Asp Asn Trp Ile Val His Pro Ser
Ser Phe

SEQ ID NO:21

Ser Ala Val Pro Trp Ser Pro Ala Ala Leu Ser Leu
Leu Gln Arg Pro Ser Glu Asp Val Ala Ala His Thr
Asn Cys Arg Arg Ala Ser Leu Asn Ile Ser Phe

SEQ ID NO:22

Ser Ala Val Pro Trp Ser Pro Ala Ala Leu Ser Leu
Leu Gln Arg Pro Ser Glu Asp Val Ala Ala His

SEQ ID NO:23

Ser Ala Val Pro Trp Ser Pro Ala Ala Leu Ser Leu
Leu Gln Arg Pro Ser Glu Asp Val Ala Ala His Thr
Asn Cys Arg Arg Ala Ser Leu Asn Ile Ser Phe Glu
Glu Leu Gly Trp Asp Asn Trp Ile Val His Pro Ser
Ser Phe Val Phe His Tyr Cys His Gly Asn Cys Ala
Glu Gly His Gly Leu Ser His Arg Leu Gly Val Gln
Leu Cys Cys Ala Ala Leu Pro Gly Thr Met Arg Ser
Leu Arg Val Arg Thr Thr Ser Asp Gly Gly Tyr Ser
Phe Lys Tyr Glu Thr Val Pro Asn Leu Leu Ala Gln
Asp Cys Thr Cys Val

SEQ ID NO:24

Leu Ser Leu Leu Gln Arg Pro Ser Glu Asp Val Ala
Ala His

SEQ ID NO:25

His Thr Asn Cys Arg Arg Ala Ser Leu Asn

SEQ ID NO:26

His Tyr Cys His Gly Asn Cys Ala Glu Gly His Gly
Leu Ser His Arg Leu Gly Val Gln

SEQ ID NO:27

Gln Leu Cys Cys Ala Ala Leu Pro Gly Thr Met Arg
Ser

SEQ ID NO:28

Ser Leu Arg Val Arg Thr Thr Ser Asp Gly Gly Tyr
Ser Phe Lys Tyr Glu Thr Val Pro Asn Ile

Other embodiments include hybrid peptides in which each amino acid residue of the hybrid peptide is identical to or a conservative substitution of the amino acid residue at the corresponding position of the PPR of inhibin or a fragment thereof.

Preferably, the amino acid sequence of a hybrid peptide comprises a subsequence from a first PPR and a subsequence from a second PPR. Each subsequence has from about 4 to about 115 amino acid residues, preferably about 4 to about 75 amino acid residues, more preferably about 4 to about 30 amino acid residues. The two subsequences can be equal in length, e.g., both subsequences can be 9-mers or 12-mers. Alternatively, the two subsequences can be of different lengths, e.g., a 12-mer and a 13-mer. One example is a hybrid peptide represented by SEQ ID NO:29, which is a 22-mer in which the first eleven amino acids correspond to a subsequence of SEQ ID NO: 1 and the second eleven amino acids correspond to a different subsequence of SEQ ID NO: 1.

SEQ ID NO:29

Ser Ala Val Pro Trp Ser Pro Ala Ala Leu Ser Pro
Ser Glu Asp Val Ala Ala His Thr Asn Cys

Another example is a peptide represented by SEQ ID NO:30, a 22-mer in which the first eleven amino acids correspond to a different subsequence of SEQ ID NO:1 and the second eleven amino acids correspond to another subsequence of SEQ ID NO:1.

SEQ ID NO:30

Val Pro Trp Ser Pro Ala Ala Leu Ser Leu Leu Pro
Ser Glu Asp Val Ala Ala His Thr Asn Cys

Another embodiment of the present invention is a peptide derivative of a hybrid peptide, for example, a peptide derivative of a peptide represented by, for example, SEQ ID NO:29 or SEQ ID NO:30. Included within the definition of “peptide derivative of a hybrid peptide” are fragments of hybrid peptides, which generally have at least about 10 amino acid residues and can affect production performance. A “peptide derivative of a hybrid peptide” also includes a peptide having a “modified sequence” in which one or more amino acids in the hybrid peptide have been substituted with a naturally occurring amino acid or amino acid analog (also referred to as a “modified amino acid”). Suitable modified sequences are those that are substantially homologous to the amino acid sequence of the hybrid peptide or to a subsequence thereof that can affect production performance. Peptide derivatives generally have between about 10 and about 30 amino acid residues.

In one aspect of the present invention, a peptide derivative of a hybrid peptide, e.g., SEQ ID NO:29 or SEQ ID NO:30, has an amino acid sequence corresponding to a subsequence of the hybrid peptide with between about 10 and about 15 amino acid residues which can affect production performance. Zero, one, two or three amino acid residues in the peptide derivative can differ from the amino acid residue(s) in the corresponding position of the subsequence of the hybrid peptide.

In another aspect of the present invention, a peptide derivative has an amino acid sequence corresponding to the amino acid sequence of the hybrid peptide or to a subsequence thereof which can affect production performance with between about 4 and about 115 amino acid residues, preferably about 4 and about 75 amino acid residues, more preferably from about 4 and about 30 amino acid residues. Zero, one, two, three or four amino acid residues in the peptide derivative can differ from the amino acid residue(s) in the corresponding position of the sequence or subsequence of the hybrid peptide.

As used herein, aliphatic groups include straight chained, branched or cyclic C₃-C₉ hydrocarbons which are completely saturated, which contain one or two heteroatoms such as nitrogen, oxygen or sulfur and/or which contain one or more units of unsaturation. Aromatic groups include carboxylic aromatic groups such as phenyl and naphthyl and heterocyclic aromatic groups such as imidazolyl, indolyl, thienyl, furanyl, pyridyl, pyranyl, oxazolyl, benzothienyl, benzofuranyl, quinolinyl, isoquinolinyl and acridinyl.

Suitable substituents on an aliphatic, aromatic or benzyl group include, for example, —H, halogen (—Br, —Cl, —I and —F), —O (aliphatic, substituted aliphatic, benzyl, substituted benzyl, aryl or substituted aryl group), —CN, —NO₂, —COOH, —NH₂, —NH (aliphatic group, substituted aliphatic, benzyl, substituted benzyl, aryl or substituted aryl group), —N (aliphatic group, substituted aliphatic, benzyl, substituted benzyl, aryl or substituted aryl group)₂, —COO (aliphatic group, substituted aliphatic, benzyl, substituted benzyl, aryl or substituted aryl group), —CONH₂, —CONH (aliphatic, substituted aliphatic group, benzyl, substituted benzyl, aryl or substituted aryl group)), —SH, —S (aliphatic, substituted aliphatic, benzyl, substituted benzyl, aromatic or substituted aromatic group) and —NH—C(═NH)—NH₂. A substituted benzylic or aromatic group can also have an aliphatic or substituted aliphatic group as a substituent. A substituted aliphatic group can also have a benzyl, substituted benzyl, aryl or substituted aryl group as a substituent. A substituted aliphatic, substituted aromatic or substituted benzyl group can have more than one substituent.

A “multivalent ligand” is a molecule having an array of peptides represented as “P” in the structural formulae presented above and throughout the present application. The term multivalent ligand is considered equivalent to the term multiple antigenic peptide (MAP) composition in the present application. Multivalent ligand or MAPs preferably contain from 2 to about 30 peptides. Each peptide is connected to a multilinker backbone, either by a covalent bond or through a linker group. Each peptide derivative and each linker or covalent bond is independently chosen. Non-limiting examples of a multivalent ligands are represented by structural formulae I-XV.

A “polypeptide multivalent ligand” is a multiple repeat polypeptide chain in which two or more peptides P are each separated by a peptide linker group. A polypeptide multivalent ligand is represented by structural formulae II and III.

At least two of the peptides, designated as P, in a multivalent ligand or the polypeptide multivalent ligand of the present invention may be hybrid peptides, peptide derivatives of hybrid peptides, peptide derivatives of PPRs, or combinations thereof. A multivalent ligand or a polypeptide multivalent ligand can also have one or more other peptides which do not significantly lower its ability to affect production performance. Preferably, however, all of the peptides in a multivalent ligand or a polypeptide multivalent ligand are hybrid peptides, peptide derivatives of hybrid peptides, and/or peptide derivatives of a PPR. Optionally, the C-terminus or the N-terminus of a polypeptide multivalent ligand can include amino acid sequences that might assist in its separation or isolation, such as hemagglutinin-antigen or a polyhistidine sequence. A multivalent ligand or a polypeptide multivalent ligand can have peptides which are derivatives of different PPRs and/or peptides which are derivatives of the same PPR but which have different amino acid sequences. A multivalent ligand or a polypeptide multivalent ligand can have peptides which are hybrids of a different pair of PPRs and/or peptides which are hybrids of the same two PPRs but which have different amino acid sequences. Similarly, a multivalent ligand or a polypeptide multivalent ligand can have peptides that are derivatives of different hybrid peptides and/or peptides which are derivatives of the same hybrid peptide but which have different amino acid sequences. The peptides of a multivalent ligand or a polypeptide multivalent ligand may all be the same.

Multilinker Backbone

A multilinker backbone, represented as “B” in the structures included herein, is a linear or branched molecule having a multiplicity of appropriately spaced reactive groups, each of which can react with a functional group in a peptide or linker. Suitable multilinker backbones are biocompatible and, after attachment of the peptide derivatives, are suitable for administration, such as parenteral or oral administration. Generally, the multilinker backbones have molecular weights less than about 20,000 atomic mass units (amu) and typically comprise between 2 to about 100 attachment sites. Not all attachment sites need to be occupied.

Reactive functional groups in a multilinker backbone serve as attachment sites for the peptides or linkers. Attachment sites are “appropriately spaced” when steric hindrance does not substantially interfere with forming covalent bonds between some of the reactive functional groups and the peptide.

Suitable reactive groups in a multilinker backbone include amines, carboxylic acids, alcohols, aldehydes and thiols. An amine group in a multilinker backbone can form a covalent bond with the C-terminal of a peptide derivative or a carboxylic acid functional group in a linker group. A carboxylic acid group or an aldehyde in a multilinker backbone can form a covalent bond with the N-terminus of a peptide derivative or an amine group in a linker group. An alcohol group in a multilinker backbone can form a covalent bond with the C-terminus of a peptide derivative or a carboxylic acid group in a linker group. A thiol group in a multilinker backbone can form a disulfide bond with a cysteine in a peptide derivative or a thiol group in a linker group. Bonds can also be formed between reactive functional groups in the multilinker backbone and appropriate functional groups in the amino acid side chains of the attached peptides, as described above. The functionality that connects each peptide to the multilinker backbone can be different, but is preferably the same for all peptides.

Examples of suitable polypeptide multivalent backbones are disclosed in Tam, Journal of Immunological Methods 196:17 (1996), the entire teachings of which are incorporated herein by reference. Suitable polypeptide multilinker backbones generally have between about 1 and about 20 amino acid residues, preferably 2 to 10, more preferably 3 to 7. As with other multilinker backbones, they typically have between about two and about twenty attachment sites, which are often functional groups located in the amino acid residue side chains. However, alpha amino groups and alpha carboxylic acids can also serve as attachment sites.

Preferred polypeptide multilinker backbones, represented by “B” in the structures presented above include polylysines, polyornithines, polycysteines, polyglutamic acid and polyaspartic acid, or mixtures thereof. Optionally, amino acid residues with inert side chains, e.g., glycine, alanine and valine, can be included in the amino acid sequence. The polypeptides can be pennant or cascading. A “pennant polypeptide” is linear. As with polypeptides typically found in nature, the amide bonds of a pennant polypeptide are formed between the alpha amine of one amino acid residue and the alpha carboxylic acid of the next amino acid residue. When n is less than 5, there are typically 0-6 amino acids between attachment sites; when n is greater than 5, there are typically 1-6 amino acids between attachment sites. A “cascading polypeptide” is branched with at least some of the amide bonds formed between the side chain functional group of one amino acid residue and the alpha amino group or alpha carboxylic acid group of the next amino acid residue. For example, at least some of the amide bonds of a cascading polylysine are formed between the epsilon amine group of a lysine residue and the carboxylic acid residue of the next lysine residue.

In one embodiment, the backbone “B” is comprised of 3 amino acids that may be Lys, Asp, Glu, Cys, Om, Gly, Ala and Val or mixtures thereof. Accordingly, embodiments of B include, but are not limited to the following:

Lys Lys Lys; Lys Asp Lys; Lys Lys Glu;
Cys Lys Lys; Lys Lys Orn; Cys Lys Cys;
Asp Lys Lys; Lys Ala Lys; Lys Val Lys;
Lys Ala Glu; Cys Val Lys; Lys Val Orn;
Cys Lys Ala;,
and
Asp Ala Lys.

Linkers (L)

Suitable linkers (L) are groups that can connect a peptide derivative to a multilinker backbone. In one example, the linker is an oligopeptide of from about I to about 10 amino acids consisting of amino acids with inert side chains. Suitable oligopeptides include polyglycine, polyserine, polyproline, polyalanine and oligopeptides consisting of alanyl and/or serinyl and/or prolinyl and/or glycyl amino acid residues. In another example, the linker is X₁—(CH₂)ₘ—X₂ or X₁-polyethylene-glycol-X₂. X₁ and X₂ are the residues of a functional group that is connected by a covalent bond to a suitable functional group residue in the multilinker backbone or peptide derivative, respectively. Examples of X₁ and X₂ include: 1) the residue of an alcohol group which forms an ester with the residue of a carboxylic acid group in the multilinker backbone or peptide derivative; 2) the residue of an amine group which forms an amide with the residue of a carboxylic acid group in the multilinker backbone or peptide derivative; 3) the residue of a carboxylic acid or aldehyde group which forms an amide with the residue of an amine in the multilinker backbone or peptide derivative; or, 4) the residue of a thiol group which forms a disulfide with the residue of a thiol group in the multilinker backbone or peptide derivative. m is an integer from 2 to about 20.

The peptides in a multivalent ligand can be connected to the multilinker backbone by covalent bonds, linker groups or a combination thereof. The linking groups can be the same or different. Preferably, every peptide in a multivalent ligand is connected to the multilinker backbone by a covalent bond. Alternatively, every peptide in a multivalent ligand is connected to the multilinker backbone by the same linking group, e.g., a glycine residue or a glycyl-glycyl dipeptide.

Multivalent ligands include 4-branch pennant polylysine trimers, indicating that 4 peptides are attached to a linear trimeric polypeptide backbone consisting of 3 lysine residues (Lys Lys Lys). The attachment is by means of a peptide bond between the C-terminus of each peptide or linking oligopeptide and the amino group in one of the three lysine side chains or the N-terminus of the polylysine. All peptides and all linkers in a given multivalent ligand may be the same or different.

A polypeptide spacer within a multilinker backbone B, shown in structural formula (II) is a peptide having from about 5 to about 40 amino acid residues. The spacers in a polypeptide multivalent ligand are independently chosen, but are preferably all the same. The spacers should allow for flexibility of movement in space for the flanking peptides P, and are therefore typically rich in small amino acids, for example, glycine, serine, proline or alanine. Preferably, peptide spacers contain at least 60%, more preferably at least 80% glycine or alanine. In addition, peptide spacers generally have little or no biological and antigenic activity. Preferred spacers are (Gly-Pro-Gly-Gly)ₓ. and (Gly₄-Ser)_{y}, wherein x is an integer from about 3 to about 9 and y is an integer from about 1 to about 8. Specific examples of suitable spacers include (Gly₄-Ser)₃

SEQ ID NO:31

Gly Gly Gly Gly Ser Gly Gly Gly Gly Ser Gly Gly
Gly Gly Ser
or
(Gly₄-Ser)₄

SEQ ID NO:32

Gly Gly Gly Gly Ser Gly Gly Gly Gly Ser Gly Gly
Gly Gly Ser Gly Gly Gly Gly Ser.

Spacers can also include from one to about four amino acids that create a signal for proteolytic cleavage.

The multivalent ligands or polypeptide multivalent ligands of the present invention can be co-administered with other pharmaceutically active agents. In one example, the multivalent ligands or polypeptide multivalent ligands are co-administered with other agents typically administered to birds.

The peptide derivatives, the multivalent ligands and or polypeptide multivalent ligands of the present invention have many utilities other than for increasing production performance. Some of these uses are discussed in the following paragraphs.

The disclosed multivalent ligands can be used to raise antibodies, both polyclonal and monoclonal, against the peptide derivatives attached thereto. Methods of raising antibodies against peptide antigens attached to a multilinker backbone are described in Tam, Proc. Natl. Acad. Sci. USA 85:5409 (1988), Tam and Lu, Proc. Natl. Acad. Sci. USA 86:9084 (1989) and Tam, Journal of Immunological Methods 196:17 (1996).

Suitable antibodies can also be raised against the peptide derivatives and hybrid peptides of the present invention by optionally conjugating them to a suitable carrier, such as keyhole limpet hemocyanin or serum albumin. Polyclonal and monoclonal antibody production can be performed using any suitable technique. Some of the peptides, peptide derivatives and hybrid peptides of the present invention are sufficiently antigenic that no carrier is required for production of antibodies. A variety of methods for producing monoclonal antibodies have been described (see e.g., Kohler et al., Nature, 256: 495-497 (1975) and Eur. J. Immunol. 6:511-519 (1976); Milstein et al., Nature 266: 550-552 (1977); Koprowski et al., U.S. Pat. No. 4,172,124; Harlow, E. and D. Lane, 1988, Antibodies: A Laboratory Manual, (Cold Spring Harbor Laboratory: Cold Spring Harbor, N.Y.); Current Protocols In Molecular Biology, Vol. 2 (Supplement 27, Summer 1994), Ausubel, F. M. et al., Eds., (John Wiley & Sons: New York, N.Y.), Chapter 11, (1991)). Generally, a hybridoma can be produced by fusing a suitable immortal cell line (e.g., a myeloma cell line such as SP2/0) with antibody producing cells. The antibody producing cell, preferably those of the spleen or lymph nodes, can be obtained from animals immunized with the antigen of interest. The cells (hybridomas) can be isolated using selective culture conditions, and cloned by limiting dilution. Cells that produce antibodies with the desired specificity can be selected by a suitable assay (e.g., ELISA).

Antibodies, including monoclonal antibodies, against the PPR of inhibin proteins and fragments thereof have a variety of uses. For example, those against or reactive with an inhibin protein, and preferably which bind specifically to the PPR of said protein, can be used to determine if the inhibin protein is present in a liquid sample obtained from a subject. The sample is treated with anti-PPR antibody specific for the inhibin protein or fragments thereof. The sample is then analyzed, for example, by Western blotting or immunoprecipitation for complexes between the inhibin protein and antibody. The sample can be, for example, a cleared lysate of a cell, which is generated for example, by treating cells with a detergent such as sodium deoxycholate (0.5%-1%) or sodium dodecyl sulfate (1%), centrifuging and separating the supernatant from the pellet.

PPRs play a key role in the biological activity of inhibin proteins, as is evidenced by the fact that multivalent ligands comprising the peptide derivatives or hybrid peptides of the present invention have such a dramatic effect on biological processes such as production performance. The polypeptide multivalent ligands, peptide derivatives and hybrid peptides of the present invention can also be used to identify molecules which interact with the PPR of specific inhibin proteins and whose activities are modulated by them. For example, an affinity column can be prepared to which a specific polypeptide multivalent ligand, peptide derivative or hybrid peptide is covalently attached, directly or via a linker. This column, in turn, can be utilized for the isolation and identification of specific molecules which bind the PPRs of inhibin proteins and which will also likely bind the inhibin protein from which the peptide derivative or hybrid peptide was derived. The molecule can then be eluted from the column, characterized and tested for its ability to interact with inhibin proteins or fragments thereof. Such peptides may be useful as peptide components of MAP compositions as taught in the present invention.

Synthesis of Peptide Sequences and MAPs

Peptide sequences in the compounds of the present invention may be synthesized by solid phase peptide synthesis (e.g., BOC or FMOC) method, by solution phase synthesis, or by other suitable techniques known to one of ordinary skill in the art including combinations of the foregoing methods. The BOC and FMOC methods, which are established and widely used, are described in Merrifield, J. Am. Chem. Soc. 88:2149 (1963); Meienhofer, Hormonal Proteins and Peptides, C. H. Li, Ed., Academic Press, 1983, pp. 48-267; and Barany and Merrifield, in The Peptides, E. Gross and J. Meienhofer, Eds., Academic Press, New York, 1980, pp. 3-285. Methods of solid phase peptide synthesis are described in Merrifield, R. B., Science, 232: 341 (1986); Carpino, L. A. and Han, G. Y., J. Org. Chem., 37:3404 (1972); and Gauspohl, H. et al., Synthesis, 5:315 (1992)).

Multivalent ligands can be prepared by methods known to one of ordinary skill in the art, including methods disclosed in Tam, J., Immunological Methods 196:17 (1996), Kim et al., Cancer Research 54:5005 (1994), Nomizu et al., Cancer Research 53:3459 (1993) and Tam, J., Proc. Natl. Acad. Sci., USA 85:5409 (1989).

A convenient method of preparing multivalent ligands in a single operation is by stepwise solid phase synthesis, starting with the C-terminus core matrix, for example, using a diprotected Boc-Lys(Boc) in Boc chemistry or Fmoc-Lys(Fmoc) in Fmoc chemistry to reach the desired branching. The selected peptide derivative, hybrid peptide or hybrid peptide derivative is then sequentially elongated to the lysinyl core matrix on the resin to form the desired multivalent ligand. This stepwise method produces multivalent ligands with a C→N orientation. Chimeric multivalent ligands having two or more different appended peptides can be also produced in this way by tandem synthesis of both sequences in a continuous array. Alternatively, different peptides can be synthesized on the different arms of the core matrix, using a core matrix bearing two different amine-protecting groups. Methods to distinguish the α and ε amines of lysines so that different peptides and functional moieties could be introduced have been developed. A common theme in these methods is the manipulation of the orthogonality or differential liability of deprotecting methods. Suitable combinations include: (i) Boc-Fmoc (Tam and Lu, Proc. Natl. Acad. Sci., USA 25 86:9084 (1989); (ii) Fmoc-Dde (Bycroft et al., J. Chem. Soc. Chem. Commun. 1993:773 (1993); and, (iii) Npys-Fmoc (Ahlborg, J. Immunol. Methods 179:269 (1995). Other procedures for preparing polypeptide multivalent ligands are disclosed in Rotzschlee et al., Proc. Natl. Acad. Sci. USA 94:1462 (1997).

Peptides to be used in the MAP compositions of the present invention may also be made and subsequently purified using recombinant techniques and purification techniques known to one of ordinary skill in the art of using expression systems to make peptides and proteins.

Preparation of a MAP protein

The synthesis of a tetra branched multilinker backbone (B) with an PPR-peptide attached was accomplished manually by a stepwise solid-phase procedure (Merrifield, R. B., J. Am. Chem. Soc., 85:2149-2154 (1963) on t-butoxycarbonyl (Boc) βAla-OCH₂-Pam resin (Mitchell, A. R., et al., J. Org. Chem. 43:2845-2852 (1978) in which 0.05 mmol of βAla is present in 0.5 g of resin. The synthesis of the first and every subsequent level of the carrier core was achieved using a 4 M excess of preformed symmetrical anhydride of N^{α}, N^{ε}-Boc-Lys(Boc) (0.2, 0.4, 0.8 and 1.6 mmol consecutively) in dimethylformamide (HCONMe₂ 12 ml/g resin) followed by a second coupling via dicyclohexylcarbodiimide alone in CH₂Cl₂ to give, after deprotection, the tetra-branched core matrix containing four functional amino groups. The protecting groups for the synthesis of the peptide antigens were Boc groups for the α-amino termini and benzyl alcohol derivatives for most side chain amino acids. For all residues except arginine, asparagine, glutamine, and glycine, the first coupling for 1 hour, monitored by quantitative ninhydrin test (Sarin, V. K., et al., Anal. Biochem., 117:147-157 (1981) was done with the preformed symmetrical anhydride in CH₂Cl₂, a second coupling in HCONMe₂, and a third (if needed) in N-methylpyrrolidone at 50° C. (Tam, J. P., in Proceedings of the Ninth American Peptide Symposium, eds. Deber, C. M., Kopple, K. D. and Hruby, V. J. (Pierce Chem., Rockford, Ill.) pp. 305-308 (1985)). The coupling of Boc-Asn and Boc-Gln was mediated by the preformed 1-hydroxybenzotriazole ester in HCONMe₂. Boc-Gly and Boc-Arg were coupled with water soluble dicyclohexylcarbodiimide alone to avoid, respectfully, the risk of formation of dipeptide and lactam. To eliminate the polycationic amino groups, which give highly charged macromolecules, the peptide chains were capped on their α-amino group by acetylation in 3 mM acetic anhydride in HCONMe₂ containing 0.3 mmol of N,N-dimethylaminopyridine at the completion of the multivalent ligand. The deprotection process was initiated by removing the dinitrophenyl protecting group of His(Dnp) with 1 M thiophenol in HCONMe₂ for 8 hours (3 times and at 50° C. if necessary to complete the reaction). The branched peptide oligolysine was removed from the crosslinked polystyrene resin support with the low-high-HF method or the low-high trifluoromethane-sulfonic acid method of cleavage to yield the crude multivalent ligand (85%-93% cleavage yield) (Tam, J. P., et al., J Am. Chem. Soc., 108:5242-5251 (1986). The crude peptide and resin were then washed with cold ether/mercaptoethanol (99:1, vol/vol., 30 ml) to remove ρ-thiocresol and ρ-cresol, and the peptide was extracted with 100 ml of 8M urea/0.2 M dithiothreitol/0.1 M Tris-HCl buffer, pH 8.0. To remove all the remaining aromatic by-products generated in the cleavage step, the peptide was dialyzed in Spectrum Por 6 tubing, 1000 MW cutoff, by equilibration for 24 hours with a deacrated and N₂-purged solution containing 8 M urea, 0.1 M NH₄HCO₃(NH₄)₂CO₃, pH 8.0, with 0.1 M mercaptoethanol at 0° C. for 24 hours. The dialysis was then continued in 8 M and then in 2 M urea—all in 0.1 M NH₄HCO₃/(NH₄)₂CO₃ buffer, pH 8.0 for 12 hours and then sequentially in H₂O and 1 M HOAc to remove all urea. The multivalent ligand was lyophilized and purified batch wise by high-performance gel-permeation or ion-exchange chromatography. All purified material was analyzed and found to contain the predicted amino acid sequences.

Other important features of the multilinker backbone (B) as an antigen carrier are that the exact structure is known; there are no contaminants which may be themselves antigenic, produce tissue irritation or other undesirable reactions; the exact concentration of the inhibin peptide(s) (P) is/are known; P is symmetrically or asymmetrically distributed on the backbone; and, the backbone can be utilized as a base for more than one antigen so that multivalent vaccines can be produced. One principal advantage of the MAP compositions of this invention as the basis for inhibin vaccines is that unlike previous systems using natural carriers such as keyhole limpet hemocyanin, maltose binding protein, tetanus toxoid and bovine serum albumin, the multilinker backbones (B) of this invention are fully defined chemical entities on which the inhibin peptide antigens are bound in known concentrations. Additionally, the inhibin peptide antigens comprise a large part of the molecule and not a relatively small and undefined proportion of the molecule as in the case of natural large carriers such as maltose binding protein.

When the MAP molecules are to be employed to produce a vaccine, it is preferred that the multilinker backbone be a naturally occurring amino acid, such as lysine, so that it can be processed by the body following the usual metabolic pathways. However, as will be explained more fully hereinafter, amino acids that are not naturally occurring, even those which are not a-amino acids can be employed. The amino acids, or any other asymmetric molecules used in building the backbone can be in either the D or L form.

Although the dendritic polymers have been principally described hereinabove as polyamide polymers, it will be readily apparent that the multilinker backbones (B) of this invention are not limited to dendritic polyamides. Any of a wide variety of molecules having at least two available functional groups can serve as multilinker backbones. Propylene glycol, for example, can serve as the basis for a polyester dendritic polymer. Succinic acid with selected glycols or amines can serve as a core molecule to generate polyesters or polyamides. Diisocyanates can be used to generate polyurethanes. The important point is that the multilinker backbone has at least two available functional groups from which identical branches can be generated by sequential scaffolding type reactions with additional molecules also having at least two available functional or anchoring groups on each branch. In a simple case in which the multilinker backbone has two available functional groups and each succeeding generation has two available functional groups, the number of anchoring sites to which peptides (P) or linkers (L) can be anchored is expressed by (2)ⁿ where n is the number of the generation.

For a more complete discussion of the chemistry of dendritic polymers attention is directed to Tomalia et al., Polymer Journal 17 (1), 117 (1985), Aharoni et al., Macromolecules 15, 1093 (1982), and the following U.S. Pat. Nos. 4,289,872; 4,376,861; 4,507,466; 4,515,920; 4,517,122; 4,558,120; 4,568,737; 4,587,329; 4,599,400; and, 4,600,535.

This invention, in its presently preferred embodiments, provides a multiple antigen peptide system comprising a multilinker backbone (B) with a plurality of anchoring sites covalently bound to peptides (P) that may be the same or different, and optionally a linker (L) between the peptide and the backbone. The multilinker backbone has at least two functional groups to which molecular branches having terminal functional groups are covalently bound. The terminal functional groups on the branches are covalently bonded to peptides (P). The antigenic molecules P are principally described herein as peptide antigens, but they are not limited to peptide antigens or even to antigens.

The selected peptides (P) may be separately synthesized or otherwise obtained and joined to the backbone (B). Alternatively, P may be synthesized on the backbone. For instance, if P is an oligopeptide or relatively low molecular weight polypeptide, and the available functional groups on B are amino groups or carboxyl groups, P can be synthesized by extending each branch of B utilizing known peptide synthesis techniques.

A particular advantage of this invention is that the B can serve as a carrier for two or more different Ps. These Ps may all be related to inhibin or may be a mixture of inhibin-related and other peptides. This is particularly useful for producing MAP vaccines, or for producing MAP vaccines that contain one or more Ps and one or more antigens useful for conferring protection against avian diseases. Vaccines produced from antigenic products of the invention in which T-cell antigens and B-cell antigens associated with the same disease are joined to the backbone polymer in any various configurations are useful because they are capable of generating high antibody titers.

One embodiment of this invention that utilizes this procedure is based on the use of a polylysine as B or other structurally similar molecule employing different amino blocking groups, one of which is stable to acid hydrolysis, the other of which is stable to alkaline hydrolysis. This makes it possible to protect either of the amino groups of lysine by the orthogonal protection method.

Fluorenylmethyloxycarbonyl (Fmoc) is a base labile protecting group and is completely stable to acidic deprotection. The t-butoxycarbonyl blocking group (Boc) is stable under basic conditions but not stable under mildly acidic conditions such as 50% trifluoroacetic acid. By choosing Boc-lys (Boc)-OH, Boc-lys (Fmoc)-OH, Fmoc-lys (Boc)-OH or Fmoc-lys (Fmoc)-OH it is possible to place one set of antigens on the alpha amino group of lysine and another on the omega amino group. Those skilled in the art of peptide synthesis can readily devise methods of achieving the same types of products using diverse blocking groups and other dendritic polymers.

The following abbreviations are used in the examples:
Boc-13 t-butoxycarbonyl
TFA—trifluoracetic acid
DMF—dimethylformamide
DCC—dicyclohexylcarbodiimide
Tos—tosyl
Bzl—benzyl
Dnp—dinitrophenyl
2CIZ—2-chlorocarbobenzoxy
DIEA—diisopropoylethylamine
TFMSA—trifluormethylsulfonic
BSA—bovine serum albumin
HPLC—high performance liquid chromatography
TBR—tumor bearing rabbit
ATP—adenosine triphosphate
Dnp—dinitrophenyl.
CIZ—chlorobenzyloxycarbonyl
BrZ—bromobenzyloxycarbonyl
ELISA—enzyme linked immunoabsorbent assay
   Methods of Enhancing Production Performance

It has been unexpectedly discovered that the compositions of the present invention enhance the production performance of animals, and in particular the production performance of birds. Accordingly, the present invention is also directed to a method of enhancing production performance in animals via the administration of the MAP compositions of the present invention together with an acceptable carrier. In one embodiment, the method comprises administering an effective amount of the MAP composition to a female animal together with an acceptable carrier such that production performance of the animal is increased. In another embodiment, the method comprises administering an effective amount of the MAP composition together with an acceptable carrier to a male animal such that production performance of the animal is increased. Preferably, an immunological response directed against the MAP composition occurs in the animal. More preferably, the immunological response that occurs in the animal is also directed against the inhibin protein produced by the animal (endogenous inhibin).

More particularly, the method of the present invention comprises the administration of an effective amount of the MAP composition of the present invention to an animal such that the production performance of the animal is enhanced. Preferably, the animal is a bird. A preferred bird is a poultry bird. Especially preferred birds are chickens, quail, turkeys, geese, and ducks. It is to be understood that a “treated” bird is a bird to which the MAP composition of the present invention has been administered.

The method of the present invention can be used to enhance production performance in any species of female bird that produces inhibin. The female bird includes, but is not limited to, a ratite, a psittaciforme, a falconiforme, a piciforme, a strigiforme, a passeriforme, a coraciforme, a ralliforme, a cuculiforme, a columbiforme, a galliforme (domestic fowl), an anseriforne (geese, ducks, other water fowl), and a herodione. More particularly, the female bird includes, but is not limited to, an ostrich, emu, rhea, kiwi, cassowary, turkey, quail, chicken, falcon, eagle, hawk, pigeon, parakeet, cockatoo, macaw, parrot, perching bird (such as, song bird, jay, blackbird, finch, warbler, sparrow), and any member of the order psittaciformes. Preferred birds are poultry. A particularly preferred poultry bird is a chicken, quail, turkey, duck or goose. The method of the present invention can also be used to accelerate the onset of egg lay in species of birds that are endangered. Such endangered birds include, but are not limited to, grouse, prairie chicken, eagles, hawks, condors, and owls.

The peptide fragments (P) of inhibin in the MAP composition of the present invention can from inhibin from any species of animal that produces inhibin. Peptide fragments (P) in a given MAP composition may be from different species. The inhibin includes, but is not limited to, bird inhibin, mammal inhibin, reptile inhibin, amphibian inhibin, or fish inhibin, among others. More specifically, the mammal inhibin includes, but is not limited to, cow inhibin, human inhibin, horse inhibin, cat inhibin, dog inhibin, rabbit inhibin, sheep inhibin, mink inhibin, fox inhibin, otter inhibin, ferret inhibin, raccoon inhibin, donkey inhibin, rat inhibin, mouse inhibin, hamster inhibin, and pig inhibin. The bird inhibin includes, but is not limited to, ostrich inhibin, emu inhibin, rhea inhibin, cassowary inhibin, kiwi inhibin, turkey inhibin, quail inhibin, chicken inhibin, duck inhibin, goose inhibin, and inhibin from members of the order psittaciformes.

A preferred inhibin is avian, or bird, inhibin. A more preferred inhibin is chicken inhibin. Most preferably, the heterologous protein of the present invention comprises alpha-subunit inhibin protein, or a fragment thereof, and a carrier protein.

The inhibin peptides, P, a fragment thereof or a substitution thereof, can be isolated from animal fluids, expressed from genetically engineered cells in an expression system, or synthetically produced from a series of chemical reactions. More particularly, the fragment of inhibin includes, but is not limited to the following compositions: alpha-subunit inhibin; β-subunit inhibin; recombinant DNA derived fragments of alpha-subunit inhibin or β-subunit inhibin; synthetic peptide replicas of fragments of alpha-subunit inhibin or β-subunit inhibin; synthetic peptide replicas of the N-terminal sequence of alpha-subunit inhibin or β-subunit inhibin; fragments of partially purified inhibin from follicular fluid; fragments of endogenous alpha-subunit inhibin or β-subunit inhibin; and fragments of exogenous alpha-subunit inhibin or β-subunit inhibin. As stated above, it is most preferable that the fragment of inhibin is the mature alpha-subunit inhibin, or a fragment thereof. By inhibin, it is understood by one of ordinary skill in the art to encompass inhibin with amino acid substitutions that might render it more immunogenic, or more active at a receptor. It is to be understood that the inhibin in the MAP composition need not be from the same species to which the MAP composition will be administered. For example, a MAP composition that is administered to an ostrich can be comprised of chicken inhibin.

Carriers

It is to be understood that the MAP composition of the present invention can further comprise carriers, vehicles, adjuvants, preservatives, diluents, emulsifiers, stabilizers, and other known components that are known and used in vaccines of the prior art.

The terms “acceptable carrier” or “acceptable vehicle” are used herein to mean any liquid including but not limited to water or saline, a gel, salve, solvent, diluent, fluid ointment base, liposome, micelle, giant micelle, and the like, which is suitable for use in contact with living animal or human tissue without causing adverse physiological responses, and which does not interact with the other components of the composition in a deleterious manner. The MAP compositions of the present invention may conveniently be presented in unit dosage form and may be prepared by conventional pharmaceutical techniques. Such techniques include the step of bringing into association the active ingredient and the carrier(s) or excipient(s). In general, the formulations are prepared by uniformly and intimately bringing into association the active ingredient with liquid carriers. Formulations suitable for parenteral administration include aqueous and non-aqueous sterile injection solutions which may contain anti-oxidants, buffers, bacteriostats and solutes; and aqueous and non-aqueous sterile suspensions which may include suspending agents and thickening agents. The formulations may be presented in unit-dose or multi-dose containers, for example, sealed ampules and vials, and may be stored in a freeze-dried (lyophilized) condition requiring only the addition of the sterile liquid carrier, for example, water for injections, immediately prior to use. Extemporaneous injection solutions and suspensions may be prepared from sterile powders, granules and tablets commonly used by one of ordinary skill in the art.

Preferred unit dosage formulations are those containing a dose or unit, or an appropriate fraction thereof, of the administered ingredient. It should be understood that in addition to the ingredients, particularly mentioned above, the formulations of the present invention may include other agents commonly used by one of ordinary skill in the art.

The MAP compositions of the present invention may be administered through different routes, such as oral, including buccal and sublingual, rectal, parenteral, ocular, aerosol, nasal, intramuscular, subcutaneous, intradermal, and topical. The MAP compositions of the present invention of the present invention may be administered in different forms, including but not limited to solutions, emulsions and suspensions, microspheres, particles, microparticles, nanoparticles, and liposomes.

Adjuvants

Any adjuvant system known to one of ordinary skill in the art may be administered together with the composition of the present invention. Such adjuvants include, but are not limited to, Freund's incomplete adjuvant, Freund's complete adjuvant, polydispersed β-(1,4) linked acetylated mannan (“Acemannan”), Titermax® (polyoxyethylene-polyoxypropylene copolymer adjuvants from CytRx Corporation), modified lipid adjuvants from Chiron Corporation, saponin derivative adjuvants from Cambridge Biotech, killed Bordetella pertussis, the lipopolysaccharide (LPS) of gram-negative bacteria, large polymeric anions such as dextran sulfate, and inorganic gels such as alum, aluminum hydroxide, or aluminum phosphate. Emulsions, including but not limited to water in oil emulsions and water in oil in water emulsions may also be used to administer the MAP compositions of the present invention. Another adjuvant system is Freund's incomplete adjuvant. Yet another adjuvant system is Freund's complete adjuvant.

Methods of Administration

The MAP composition of the present invention can be administered with an acceptable carrier to a bird by any means known in the art. For example, the composition can be administered subcutaneously, intraperitoneally, intraocularly, intradermally, or intramuscularly. Preferably, the composition is injected subcutaneously. The composition can be administered to the bird in one or more doses. Preferably, the composition is administered to the bird in multiple doses wherein an initial immunization is followed by booster immunizations.

The composition can be administered to an animal at any time before the animal ceases to ovulate or produce sperm due to disease or age. The preferred age at which the composition of the present invention is administered to an animal depends upon the species of the animal involved, the mating season (if any) of an animal, and upon the purpose of the administration of the composition.

For example, where the composition is administered to accelerate the onset of egg lay or sperm production, the composition of the present invention is to be administered to a bird before the bird reaches the normal age of egg lay or puberty. As stated above, the preferred age at which the composition of the present invention is first administered to an animal depends upon the species of the animal involved, the mating season (if any) of an animal, upon the size of the bird, and upon the identity of the inhibin peptide fragments in the composition.

As another example, where the composition is administered to enhance production performance of agricultural animals that have breeding seasons, the preferred time of administering the composition is prior to the start of the breeding season. In contrast, where the composition is to be administered to a mature animal that has a suppressed egg production rate or a suppressed sperm production rate, then the composition would be administered at the time that the suppression is recognized as problematic.

The preferred timing of administration to chickens is after the birds reach immunocompetence but before they reach reproductive maturity. The preferred minimum time between primary and booster injections is about 3 weeks. In one embodiment, a primary injection and a booster injection occur at about 15 and 18 weeks, respectively. When composition is administered to accelerate the onset of egg lay or sperm production in chickens, the preferred time of administering the composition is to have a primary and one or more inoculations administered prior to 20 weeks-of-age in meat-type chickens (broiler breeder pullets) and 18 weeks-of-age in egg-type chickens (e.g. Leghoms).

With respect to an animal having a breeding season, although the MAP composition of the present invention can be administered to a bird at any age, immunizing the bird during the six months prior to the bird's first breeding season is preferable. It is understood by those of ordinary skill in the art that average female birds initiate egg lay during the first breeding season. It is even more preferable to immunize the bird approximately two to six months prior to the bird's first breeding season, and then to administer booster immunizations at one-month intervals prior to the bird's first breeding season. It is most preferable to immunize the bird approximately six months prior to the bird's first breeding season, and then to administer a booster immunization one month prior to the bird's first breeding season.

The primary immunization comprises between approximately 0.005 to 1.0 mg of the MAP composition of the present invention. The booster immunizations comprise between approximately 0.0025 to 1.0 mg of the MAP composition of the present invention. Preferably, the primary immunization comprises between approximately 0.01 to 0.75 mg of the MAP composition of the present invention. More preferably, the primary immunization comprises between about 0.02 to 0.50 mg of the MAP composition of the present invention. The booster immunizations preferably comprise between approximately 0.005 to 0.5 mg, more preferably between about 0.01 to 0.025 mg of the MAP composition of the present invention. It is also preferable that the MAP composition is emulsified in a water in oil (WO) or water in oil in water (WOW) emulsion in the primary immunization, and that the MAP composition is emulsified in a water in oil (WO) or water in oil in water (WOW) emulsion in the booster immunizations. Preferably, the MAP composition is injected subcutaneously. Preferably, the MAP composition is injected subcutaneously at one site along the back of the neck region of the animal, preferably a bird.

The amount of the MAP composition of the present invention administered to a bird varies according to the species of the bird, the age and weight of the bird, when the composition is administered in relation to the breeding season (if the bird has a breeding season), and how many times the composition is to be administered. Also, the commencement of the administration schedule, or treatment schedule, varies according to the species of the bird, the average age of puberty of that species of the bird, the family history of the bird (with respect to the family's history of age at puberty), the time of year the bird was hatched, the nutritional plane of the bird (well nourished birds come into puberty before those that are undernourished), the bird's body weight, the degree of fleshing and fattening, ambient lighting conditions (with respect to natural and artificial housing as these factors interact with light intensity and duration of the daily photoperiod), the general health of the bird at that time of commencement, immunological competence of the bird, the long term health history of the bird, the presence of extreme weather conditions (prolonged excessive inclement weather such as rain, heat, or windiness that the bird is not accustomed to), housing conditions (overcrowding), and a lack of exercise.

One of ordinary skill in the art, in view of the teachings of the present invention, would be able to determine by routine testing the amount of MAP composition that will be necessary to elicit an immunological response to the protein by the bird.

Another example of the method for enhancing production performance is as follows. An immunologically effective amount of the MAP composition is administered to a mammal such that an immunological response occurs in the mammal that is directed against the MAP composition. The MAP composition is preferably comprised of peptide fragments of mammalian inhibin linked to an amino acid backbone. Another preferred MAP composition is comprised of peptide fragments of avian or reptilian inhibin.

For example, the following is a brief summary of the method of the present invention for enhancing production performance in Japanese Quail as fully discussed in Example 4. The average age at puberty for an untreated quail is approximately six to eight weeks. The following is a treatment schedule for Japanese quail having an approximate body weight range of 0.1 to 0.25 pounds: primary (first) injection of between about 0.025 to 0.1 mg of the MAP composition of the present invention on its 25th day of age; and a booster of 0.01 to 0.05 mg between 32 to 35 days of age.

The following is a brief summary of the method of the present invention for enhancing production performance in chickens as discussed in Example 2. The average age at puberty for an untreated egg-type chicken (e.g., Leghorn) is approximately 20 weeks. The following is a treatment schedule for a egg-type chicken having an approximate body weight range of 2.0 to 3.0 pounds: primary (first) injection of about 0.05 to 0.1 mg of the MAP composition of the present invention at the 14th week of age and a booster of 0.025 to about 0.05 mg at the 17th week of age. The average age at puberty for an untreated meat-type chicken (broiler breeder) is approximately 23-25 weeks. The following is a treatment schedule for a meat-type chicken having an approximate body weight range of 3.0 to 4.0 pounds: primary (first) injection of between about 0.05 to 0.1 mg of the MAP composition of the present invention at the 15th week of age and a booster of about 0.025 to about 0.05 mg at the 18th week of age.

The following is a brief summary of the method of the present invention for enhancing production performance in turkeys as is discussed in Example 5. The average age at puberty for an untreated turkey is approximately 30 weeks. The following is a treatment schedule for a turkey having an approximate body weight range of 9.0 to 12 pounds: primary (first) injection of about 0.1 to about 0.5 mg of the MAP composition of the present invention on its 24th week of age and a booster of between about 0.05 to about 0.25 mg on the 27th week of age.

As discussed above, the method of the present invention enhanced production performance by accelerating the onset of puberty in the animal that the composition of the present invention was administered to. The term “accelerates” with respect to the onset of egg lay denotes that egg lay of a treated bird commences at least about 3% earlier than egg lay would ordinarily commence in an untreated bird. Preferably, egg lay commences at least about 5% earlier, and more preferably commences at least about 7% earlier. Even more preferably, egg lay commences at least about 10% earlier, and most preferably commences at least about 13% earlier than egg lay would ordinarily commence in an untreated bird.

Also, as discussed above, the method of the present invention enhanced production performance by increasing egg or sperm production intensity in animals. The term “increases” with respect to egg production denotes that egg production of a treated bird increases at least about 3% with respect to the amount of egg production in an untreated bird. Preferably, egg production increases at least about 7%, and more preferably increases at least about 12%.

Further, as discussed above, the method of the present invention enhances production performance by accelerating the onset of maximum egg production in an animal. The term “accelerates” with respect to the onset of maximum egg lay denotes that maximum egg lay of a treated bird commences at least about 3% earlier than egg lay would ordinarily commence in an untreated bird. Preferably, maximum egg lay commences at least about 5% earlier, and more preferably commences at least about 7% earlier. Even more preferably, maximum egg lay commences at least about 10% earlier, and most preferably commences at least about 13% earlier than maximum egg lay would ordinarily commence in an untreated bird.

Surprisingly, the composition of the present invention is useful to increase the lifetime total egg lay of birds. The term “increase” with respect to total lifetime egg lay denotes that the total lifetime egg lay of a treated bird increases at least about 3% with respect to the total lifetime egg lay of an untreated bird. Preferably, total lifetime egg lay increases at least about 7%, and more preferably increases at least about 12%. Most preferably, total lifetime egg lay increases at least about 15%.

Unexpectedly, the composition of the present invention is useful to decrease or eliminate the need to molt a female bird, e.g., to prolong egg-laying persistency by providing for a second cycle of lay. More particularly, if the composition described above is continually administered to the female bird, as disclosed in the method above, the rate of egg lay of the bird, in comparison to if the bird was not treated with the composition of the present invention, would remain high enough so that the bird would not need to be molted to improve its rate of egg lay. It is a common practice in the art to molt a female bird, such as chicken hens (Single Comb White Leghorns, table egg producers), when its egg lay production declines such that the economic cost of maintaining the bird outweighs the economic benefit yielded by the eggs produced. To “molt” a chicken hen, the bird typically undergoes a period of fasting of approximately four to fourteen days until it beings to molt, e.g., lose its feathers. (there are alternate methods for inducing a molt, e.g. decreasing the lighted portion of their daily light:dark cycle.) During the molting period, the bird stops laying eggs. After the bird is placed back onto normal levels of feed, egg production recommences after a period of time. The entire molting period is approximately two months from the beginning of the fast period to the onset of the next egg-lay cycle. In effect, the egg production rate of the bird is rejuvenated. However, after molting a chicken, its rate of egg-lay in the next cycle does not equal the egg production during the first (pre-molt) egg-lay cycle. M. North and D. Bell, Commercial Chicken Production Manual, fourth edition, Chapter 19, Published by Van Norstrand Reinhold of New York.

For example, chickens that are used to produce table eggs reach egg lay at approximately 20 weeks, and produce an economically significant number of eggs for approximately 40 to 50 weeks. At the peak of egg lay, chickens produce eight to nine eggs every ten days. However, after approximately 50 weeks of egg lay, the rate of egg production decreases to approximately 60% of peak egg lay. At this point, the cost of the feed for the chicken is greater than the value of the eggs its produces. It is common practice to molt the chicken at this point, so that when the chicken recommences egg lay, its rate of egg lay is increased. By “prolonging the persistence of egg lay” with reference to chickens and quail, among other birds, it is meant that egg lay will be prolonged for approximately one to four weeks.

Therefore, the composition of the present invention, as it maintains the rate of egg lay at a higher level than if the bird were not treated with the composition, reduces or eliminates the need to molt a bird. The reduction or elimination of the need to molt a bird results in significant savings. More particularly, during the period that a bird is molted, and prior to that time, the bird has been unproductive with respect to its feed cost before it is molted, and then it is unproductive for a period of time after feeding recommences. Maintaining the rate of egg lay at an enhanced level therefore eliminates or reduces these unproductive phases of the bird, thereby reducing the producer's costs and increasing the producer's profits. Maintaining the rate of egg lay at an enhanced level further enhances egg producer's profits as the rate of egg-lay after molting does not equal the rate of egg-lay in the first cycle of egg lay as discussed above.

Briefly described, the rate of egg lay of birds is enhanced, thereby avoiding the need to molt the bird, by administering an effective amount of the MAP composition of the present invention to induce an immunological response thereto, and thereafter administering an effective amount of the MAP composition (boosters) to maintain a higher than normal rate of egg lay.

The method of the present invention enhances production performance in female animals that produce inhibin, such as mammals, reptiles, and birds such as poultry. More particularly, this method enhances production performance in chickens, quail, ducks, geese, and turkeys. Unexpectedly, the method of the present invention increases the onset of puberty or first egg lay in animals. Also, the method of the present invention accelerates the onset of maximum egg lay in an animal. Further, the method of the present invention increases the number of eggs laid by an animal. Further still, the method of the present invention prolongs the persistence of maximum egg lay in animals. Still further, the method increases the lifetime total egg lay of an animal. In avians, the method of the present invention also improves the feed conversion ratio of the bird. Also, the method of the present invention unexpectedly reduces or eliminates the effect of adverse laying conditions on egg lay rates of animals exposed to such conditions. Such adverse conditions include, but are not limited to, elevated temperatures, overcrowding, poor nutrition, and noise.

The immunization of an animal with the MAP composition of the present invention induces the animal to produce antibodies selectively directed against the inhibin-related peptides in the MAP composition. Preferably, the immunization also induces the animal to produce antibodies selectively directed against endogenous inhibin. While not wanting to be bound by the following statement, it is believed that the production of such antibodies by a bird reduces the time to the onset of puberty or egg lay. While not wanting to be bound by the following statement, it is believed that the production of such antibodies by the animal also enhances the animal's egg production capability or sperm production capability as the antibodies affect or neutralize the biological activity of inhibin in the animal's blood stream.

Unexpectedly, the method of the present invention also improves production performance in male animals that produce inhibin, such as mammals, reptiles, and birds. More particularly, the method of the present invention increases body weight and blood testosterone levels in male animals. Similarly, the method of the present invention increases the onset of puberty or sperm production in male animals. Also, the method of the present invention accelerates the onset of maximum sperm production in a male animal. Further, the method of the present invention increases the intensity of sperm production (sperm count) by a male animal. Further still, the method of the present invention prolongs the persistence of maximum sperm production in animals. Also, the method of the present invention increases ejaculate volume in male animals. Further, the method improves sperm viability and quality (e.g., motility) in animals. Still further, the method unexpectedly reduces or eliminates the effect of adverse conditions on sperm production of animals exposed to such conditions. Such adverse conditions include, but are not limited to, elevated temperatures, overcrowding, poor nutrition, and noise. The method of the present invention also unexpectedly increases the libido, and therefore, the reproductive potential, of a male bird.

The following examples will serve to further illustrate the present invention without, at the same time, however, constituting any limitation thereof. On the contrary, it is to be clearly understood that resort may be had to various other embodiments, modifications, and equivalents thereof which, after reading the description herein, may suggest themselves to those skilled in the art without departing from the spirit of the invention and/or the scope of the appended claims.

### EXAMPLE 1

### Production of a MAP Composition Shown in Formula XIV Using the N-Terminal 26 Amino Acids of the Mature Alpha Subunit of Chicken Inhibin (SEQ ID NO: 1)

This antigen was produced by Midwest (St. Louis, Mo.). SEQ ID NO:1 was chemically synthesized and four such sequences were covalently attached to a lysine backbone (Lys Lys Lys) to make the structure shown in formula XIV. The composition represented in formula XIV was synthesized using solid phase methods known to one of ordinary skill in the art of peptide synthesis (methods according to Merrifield, R. B., J. Am. Chem. Soc. 86, 1385 (1963) Stewart, J. M. and Young, J. D. “Solid phase peptide synthesis” Pierce Chemical Co., Rockford Ill., 1983). The lysine backbone (B) was synthesized first, and then the 26-mer amino acids (P-SEQ ID NO: I) were added onto B one at a time (starting from the C-terminal end). The entire MAP complex shown in formula XIV was cleaved from the solid phase and then lyophilized for storage

### EXAMPLE 2

### Administration of the MAP Composition Shown in Formula XIV to Broiler Breeder Female Chickens to Enhance Production Performance

Broiler breeder females were immunized with the MAP composition shown in formula XIV to generate anti-inhibin antibodies and affect production performance.

Procedure Summary

One-hundred thirty broiler breeder females (Cobb 500s) were obtained from a commercial source at 13 weeks of age. Pullets were equally assigned to 10 pens. Pens are a combination of approximately 50 sq. ft. of slat space (coated wire, Riverdale Mills) and approximately 50 sq. ft of litter (wood shavings). At 15 weeks of age, 40 pullets (4 birds/pen) were randomly selected, weighed, wing-banded and designated as test subjects.

A drip nipple watering system (6 nipples per pen) was suspended above the slats (close to the center of the slats) and extended across the slats in a direction perpendicular to the rear pen wall. Two tube-type feeders were hung above the slats on each side of the drip nipple waterer. Each feeder was located approximately equidistant between the waterer and its adjacent wall. Water was provided ad libitum by the watering system described above.

Beginning at bird placement, the 4:3 feed restriction method recommended by Cobb-Vantress was used. Beginning at 13 wk of age, birds were weighed weekly to determine if target body weights were being achieved. Feed amounts were adjusted so as to achieve target body weights. A standard broiler breeder developer mash (LSU BBD2) was fed commensurate with placement in the house. On fed days, feeding began at about 07:00 h.

Prior to the initiation of egg lay by increasing the daily photoperiod to 16 h of light: 8 h of dark (see below), pullets were maintained on a daily photoperiod of 8 h of light (i.e., from 13-20 weeks of age). During the dark period, light intensity was less than 0.5 lux. During the light period, supplemental light, supplied by incandescent bulbs, was approximately 18±0.5 lux (measured at night at bird head height). At 20 weeks of age birds were “lighted” by giving them an additional 5 h of light daily. Thereafter, 1 h per week increases in the daily photoperiod were applied until a 16 h light: 8 h dark cycle was achieved (according to recommendations of Cobb-Vantress). Beginning at 20 weeks of age, birds were also fed a standard broiler breeder layer mash (LSU BBL).

Routine observations and recording of bird and house conditions were made according to AABL SOPs # AM3-0, Daily Observations; # FR5-1, House Operations; # AM7-0, Bird Removal and # AM8-0, Chicken Gross Necropsy.

The immunological reaction of broiler breeder hens to challenge with one of three doses of the MAP complex shown in formula XIV (¹⁻²⁶INH-MAP) or Freund's (control) was tested. Within each pen, birds selected for test were given either a control challenge or one of three different dosages of the MAP composition shown in formula XIV as a primary inoculation according to the treatment schedule below (See Protocol, below). The residual birds in each pen (n=9, birds not treated with this MAP composition or serving as a control) were not considered as test animals. The primary inoculations were given at 15 weeks of age. The injection route was subcutaneous (sc) and injection vehicle was Freund's Complete Adjuvant (FCA). A single booster injection of half the primary dose was administered at 18 weeks of age. Blood samples were obtained at 19, 20 and 21 weeks of age and assessed for the presence of anti-inhibin antibodies (titer). Measures of reproductive performance were also made as follows. Estimates of the onset of puberty were made by periodic measurement of the average spread of the pubic bones of each hen (PS) as well as from calculations of the average ages at first egg lay (FIRST) and 25% egg production (T-FIVE). Mean weekly and cumulative hen-day egg production (HDEP) was also determined for 19 weeks of lay. Body weight gain (BWG; expressed as the difference between body weight at the time of primary challenge and at the end of the trial) and mortality (MORT) were also examined. Each treatment group was composed of 10 hens. Thus, including the Freund's control group, 40 birds were placed in 4 treatment groups as follows:

TABLE 1
Vaccination Protocol

| | Primary | Booster |
|---|---|---|
| Treatment | (mg/bird) | (mg/bird) |
| Control | 0 (FCA) | 0 (FIA) |
| Formula XIV | 0.05 | 0.025 |
| Formula XIV | 0.10 | 0.050 |
| Formula XIV | 0.20 | 0.100 |

The primary inoculations were given at 15 weeks of age. The injection route was subcutaneous (sc) and injection vehicle was Freund's complete adjuvant (FCA). A booster injection of one half the amount of the primary was administered at 18 weeks of age. Booster injections were also sc and the vehicle was Freund's incomplete adjuvant (FIA). Control birds received FCA as a primary inoculation (sc) at 15 weeks of age and FIA as a booster inoculation (sc) at 18 weeks of age. The three groups treated with different dosages of the MAP composition (formula XIV) and the control treatment group were identified by wing band number and not by dosage in order to blind the study to field staff. The Study Director assigned dosage amounts to wing band codes, as per AABL SOP #FR2-0, Random Allotment of Leg/Wing Badge Codes to Treatment Groups. All treatments (including the control) were represented in all pens by one bird per treatment group per pen for a total of 10 birds per treatment. The residual birds in each pen (n=9, birds not treated with the MAP compositions (formula XIV) or serving as a control) were not considered as test animals. Treatment groups were assigned as specified in AABL SOP # FR3-0, Allotment of Treatments to Broiler Breeders within Pens. At the time of treatment application, the body weights of all hens were measured according to AABL SOP # AM4-0, Animal Weighing—Broiler Breeders. Treatments were administered as specified in AABL SOP # FR4-0, Broiler Breeder Subcutaneous Injection Protocol.

Blood samples were obtained from each test hen at 19, 20 and 21 weeks of age (one, two and three weeks after the booster inoculations). Blood collections were performed as described in AABL SOP # FR6-0, Blood Collection and processed as per AABL SOP # LE4-0, Blood Processing. At the end of the trial, body weights of all hens were measured as specified in AABL SOP # AM4-0, Animal Weighing—Broiler Breeders.

From about 23 to 28 weeks of age, a total of 8 PS measures were made on all birds on test. The first six PS palpations occurred at a frequency of about every 4 days and the last two were separated by about 7 days. These measures were averaged within a bird and each bird's PS average was used as an observation for statistical analyses (see below). Daily mortality records were maintained and all birds that expired during the trial were examined for abnormalities, pathologies and probable cause of death.

Titer differences between injection treatments (0, control and the three MAP (formula XIV) dosages) were assessed using an ANOVA that incorporated a split-plot design with treatment considered on the main plot and time of blood sampling (the repeated measure; 19, 20, and 21 weeks of age or 1, 2 and 3 weeks post-booster inoculation) and the interaction of treatment with time considered on the split plot. Differences in PS, FIRST, T-FIVE, cumulative HDEP, BWG, and MORT between treatments were assessed using an ANOVA that incorporated a completely randomized design that considered the main effect of injection treatments. Where appropriate and to partition differences in these variables, treatment group means were post-hoc tested with Duncan's new multiple range test.

Measures were obtained of the onset of puberty, including PS, FIRST, and T-FIVE. Egg production was assessed by calculation of weekly HDEP and cumulative HDEP from daily observation of egg lay for 19 weeks of lay. Body weight gain (BWG; expressed as the difference between body weight at the time of primary challenge and at the end of the trial) and MORT were also determined. Comparisons were made between the three doses of the antigen and the control.

Anti-Inhibin Antibody Titers (Titer)

An ANOVA examination of the effect of injection treatments on antibody titers showed marked differences (P<0.0003) between the treatment groups. Post-ANOVA Duncan's testing to partition these differences (see below) demonstrated increased (P<0.01) mean TITER responses in birds given the lowest (0.05 mg/bird, primary) and highest (0.20 mg/bird, primary) ¹⁻²⁶INH-MAP dosages when compared to the average control TITER response. Birds inoculated with the intermediate ¹⁻²⁶INH-MAP dose (0.10 mg/bird, primary) also exhibited a robust (nearly 30-fold higher than control) mean TITER response that, despite being statistically similar to elevated TITERs seen in birds given the other two antigen dosages, failed to statistically partition from the controls. Low sampling size provides a likely explanation for this statistical oddity. As expected, largely due to the influences of the antigen treatments, TITER decreased (P<0.02) with time after booster challenge (data not shown). There were no significant interactions between injection treatment and time after booster inoculation on mean TITERs.

Measures of Puberty:

Estimates of the onset of puberty were made from periodic measures of the average spread of the pubic bones of each hen (PS), as well as from calculations of the average ages at first egg lay (FIRST) and 25% egg production (T-FIVE).

Pubic Spread (PS) Pelvic bone spacing, pubic spread or PS is a standard measurement made by the poultry industry to assess the state of sexual development in pullets prior to egg lay. Typically, PS increases from one finger width (approximately 2 cm) from 17 weeks of age to three fingers width (approximately 5 cm) at the point of lay (23-25 weeks of age), Ross Breeder Management Guide, 2000, p. 40. From about 23 to 28 weeks of age, a total of 8 PS measures were made on all birds on test. The first six occurred at a frequency of about every 4 days and the last two were separated by about 7 days. The first four PS measures occurred during the two weeks before the initiation of egg lay and the last four were conducted during a 2.5-week period thereafter. Thus, the PS measurements were essentially made during a range in flock age that spanned from the time of observation of the first egg laid by any hen in the study (a low dose ¹⁻²⁶ INH-MAP bird; 25 weeks, 3 days of age) through the first 2.5 weeks of lay. PS measures were averaged within a bird and each bird's PS average was used as an observation for statistical analyses. Mean PS was similarly increased (range: 17-38 %; P <0.01) in birds given all three doses of ¹⁻²⁶INH-MAP (formula XIV) when compared to the control PS response

TABLE 2
Pubic Spread

| ¹⁻²⁶INH-MAP | Pubic Spread |
|---|---|
| (mg/bird) | (fingers) |
| 0 | 2.64 a |
| 0.05 | 3.45 b |
| 0.1 | 3.41 b |
| 0.2 | 3.10 b |

Values with different letters a, b are different at a level of p < 0.01

Because increases in PS are considered to be indicative of a pullet's preparation for the onset of egg lay, PS measured herein (an average within each bird over the eight periods of palpation) was correlated with cumulative HDEP for the first 4 weeks of lay. PS was consistently highly correlated with early egg lay for correlations made within each injection treatment (r²=0.91, P<0.01 for the correlation within controls only and r²=0.91, P<0.01, r²=0.65, P<0.08, r²=0.85, P<0.002 within the lowest, intermediate, and highest doses of ¹⁻²⁶INH-MAP, respectively) and for the correlation that considered all birds in the study (r²=0.83, P<0.0001).

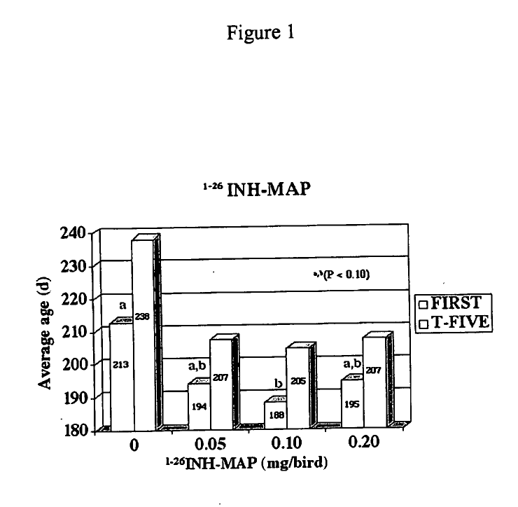

FIRST and T-FIVE When compared to the control responses, FIRST and T-FIVE measures were numerically decreased in birds given each of the three doses of ¹⁻²⁶INH-MAP and the intermediate dose group mean FIRST response was statistically different from the control response at the level of P<0.10 (FIG. 1). Low sample size undoubtedly precluded the finding of more statistical differences in these measures of puberty. Nevertheless, these measures of advancing the average age of the onset of puberty are among the best we have produced in any study. Furthermore, the present FIRST and T-FIVE findings well support the PS findings that documented dramatic increases in the average spread of the pubic bones in birds treated with ¹⁻²⁶INH-MAP (see discussion above).

Measures of Egg Lay

Weekly HDEP. During the eighth week of lay, the two highest doses of ¹⁻²⁶INH-MAP showed a greater (P<0.10) mean HDEP than that found in the controls, and the lowest ¹⁻²⁶INH-MAP treatment group had an intermediate HDEP response that was approximately 20% higher than the control response albeit not statistically different from it or the other two ¹⁻²⁶INH-MAP treatments. However, beginning at 9 weeks of lay and thereafter (until the end of the trial), marked and statistically relevant elevations (ranging from P<0.05 to 0.01) in weekly HDEP of one or more ¹⁻²⁶INH-MAP dosage groups were evident during 8 of the 11 weeks examined. Specifically, these weeks of lay were: 9, 10, 12, 13, 14, 15, 16, and 19.

Cumulative HDEP. An analysis of mean cumulative HDEP (19 weeks of lay) showed that overall egg lay was dramatically enhanced (P<0.05) in all three ¹⁻²⁶INH-MAP groups when compared to the control response (see below). The cumulative HDEP advantage over the controls for birds treated with the lowest, intermediate, and highest dose of ¹⁻²⁶INH-MAP was approximately 49%, 62%, and 64%, respectively.

TABLE 3
Cumulative Hen-day Egg Production (%)

| ¹⁻²⁶INH-MAP | 19 weeks |
|---|---|
| (mg/bird) | (mean + S.D.) |
| 0 | 40.7 + 10.8 a |
| 0.05 | 60.5 + 8.5 b |
| 0.1 | 66.1 + 4.6 b |
| 0.2 | 66.6 + 2.3 b |

Values with different letters a, b are different at p < 0.05

Measures of Body Weight Gain and Mortality

BWG. Body weight gain from the time of primary challenge (15 weeks of age) to the end of the trial (19 wk of lay) did not differ between the control and 1-26INH-MAP groups (data not shown). This indicated that treatment with the inhibin-based vaccine did not alter salvage value of the hens as measured at the mid point (19 weeks) of a full cycle of lay (40 weeks).

MORT. Mortality was also not affected by vaccine treatment (data not shown). These findings support the contention that active immunization against inhibin does not affect livability.

### EXAMPLE 3

### Administration of the MAP Complex Shown in Formula XIV to Broiler Breeder Female Chickens to Enhance Production Performance

A second major clinical trial, similar to the trial described in Example 2, was conducted in chickens.

Animals and Husbandry At 13 weeks of age, 400 Cobb-500 pullets (350 test animals and 50 extras, see below), selected at a commercial farm to be within ±10% of their recommended 13 week target body weight, were randomly placed into 16 pens. At 15 weeks of age, 350 of these pullets (14 pens) were designated as test subjects and assigned permanent individual bird identification by installation of distinctly colored and numbered wing badges. The 50 residual birds that were initially housed at 13 weeks (2 pens of 25 birds each) were maintained along with the pullets intended for study. A few of the extra birds were used for replacement of test birds that either died during 13 to 15 weeks of age (prior to treatment application) or were culled at the time of primary injection (15 weeks of age, see below). No further replacements of test animals occurred once primary inoculations were administered.

The pens and watering system were as described in Example 2. A total of 18 individual trapnests were arranged in a 2-tier system along the back wall of each pen.

Water was provided ad libitum throughout the trial. Beginning at bird placement (13 weeks of age), an everyday (ED) limited feeding method of feed restriction recommended by Cobb-Vantress was used. From 13 weeks of age until the initiation of egg lay (at 23 weeks, 5 days of age), birds were weighed weekly to determine if target body weights were being achieved. Feed amounts were adjusted so as to achieve industry recommended target body weights.

Commercially formulated and mixed broiler breeder rations (GMP certified; NAMCO, OH) were fed throughout the experiment. A grower/developer ration was fed beginning with pullet placement at 13 weeks of age until the Wednesday following 5% hen day egg production by the flock at which time the ration was changed from grower/developer to breeder. Daily feeding began at about 30 min after lights-on (see below).

Because the pullets were being reared under dark-out conditions with an 8 h daily photoperiod when they were selected at the commercial facility (at 13 weeks of age), they were continued on this regime (8 h light: 16 h dark cycle) until 20 weeks, 5 days of age. During the dark period, light intensity was less than 0.5 lux. During the light period, supplemental light, supplied by incandescent bulbs, was approximately 15±0.5 lux (measured at night at bird head height). At 20 weeks, 5 days of age, the daily photoperiod was increased by 5 h (from 8 to 13 h light daily; 60 lux). The daily duration of light was then increased by 1 hr at 21 weeks, 5 days of age and by 30 min weekly thereafter until a 16 h light: 8 h dark cycle was achieved (i.e., at 25 weeks, 5 days of age).

Routine observations and recording of bird and house conditions were made as described in Example 2. During the study it was recognized that a bird(s) might have to be removed from the study due to unanticipated events, including bird injury, sickness, etc. Therefore, removal of any and all birds from the study followed procedures described in Example 2. In addition, veterinary reports of necropsy results stating probable cause of death were obtained on all mortalities.

Injection Treatment Dosage Amounts and Schedule of Administration The test antigen used was the first 26 amino acids of chicken alpha-subunit inhibin in the form of an 4 mer multiple antigenic peptide (MAP) (¹⁻²⁶INH-MAP, formula XIV). In addition to a vehicle control (see below), the following two test article treatment dosages were administered.

TABLE 4
Vaccination Protocol

| | Primary | Booster |
|---|---|---|
| Test Article | (mg/bird) | (mg/bird) |
| ¹⁻²⁶INH-MAP | 0.05 | 0.025 |
| ¹⁻²⁶INH-MAP | 0.10 | 0.05 |

Each bird given a test article inoculation (primary and booster) was injected with an injection volume of 1.0 mL (appropriate concentration of test article in 0.5 mL of an aqueous solution +0.5 mL of the appropriate adjuvant; see below). Aqueous solution for ¹⁻²⁶INH-MAP was prepared using 5% dextrose. The primary injection vehicle for test articles and control inoculations was Freund's Complete Adjuvant (FCA). Primary inoculations (subcutaneous (sc)) were performed at 15 weeks of age. Booster injections (one half of the concentrations used in the test article primary challenges; see above) were administered at 18 weeks of age. The booster injection route was also se and the injection vehicle was Freund's Incomplete Adjuvant (FIA). Controls were given: a sc primary injection at 15 weeks of age consisting of 0.5 mL FCA +0.5 mL distilled water and a sc booster at 18 weeks of age consisting of 0.5 mL FIA +0.5 mL distilled water.

At the time of initial treatment applications (primary injections at 15 weeks of age), the flock was culled to 350 pullets and distributed equally between 14 pens (25 pullets/pen) according to established protocols. Injection treatment groups were identified by wing badge color and number and not by dosage in order to blind the study to field staff. All injection treatments were applied to appropriate birds within all pens: 5 birds per treatment group per pen for a total of 70 birds per treatment. Protocols were as described in Example 2.

Blood Collection for Anti-Inhibin Antibody Determination Blood samples were obtained from each hen at 18 weeks, 4 days and 20 weeks of age (i.e., at 4 days and 2 weeks after the booster inoculations). Blood collections were performed as described in Example 2.

Nest Habituation and Period of Egg Lay In order to minimize the acts of hens laying eggs on the floor (either in pen scratch areas or on the wire slats), pullets were habituated to use trap nests prior to the onset of egg lay. Habituation occurred daily from 21 weeks, 1 day of age to 21 weeks, 4 days of age and again from 22 weeks, 1 day of age to 22 weeks, 4 days of age. Thus, hens were finished with nest habituation training about 1 week before the observation of the first oviposition.

The egg lay period of the study began upon observation of the first oviposition by any bird. This occurred at a flock age of: 23 weeks, 5 days. The study ended after a period of 16 weeks of lay (at approximately 40 weeks of age).

Variables Measured

Puberty Measures:

Pubic Spread To determine the state of female sexual development, weekly measurements of the spacing of the pelvic (“pin”) bones were made on each hen from 17 to 26 weeks of age, according to procedures described in Example 2. Pubic spread (PS) was measured in increments of halves of fingers ranging from 1 finger (no sexual development) to 5 fingers (maximum sexual development).

FIRST and TFIVE The average age (days) at first egg lay (FIRST) was calculated for hens of each treatment, as well as the average age (days) at which individuals reached 25% egg production (TFIVE).

Hen-day Egg Production Routine egg collection (minimum of 4 times daily) began upon observation of the first trap-nested egg collected from any bird. Beginning then, and until the end of the study (16 weeks of lay), daily egg lay was recorded. Daily egg data was used to calculate weekly as well as cumulative hen-day egg production (HDEP).

Egg Weight (EWT) To assess the effects of vaccine treatments on early egg size, the first 12 eggs laid (trap-nested) by each hen of each injection treatment were individually weighed. Eggs from individual hens were collected and weighed beyond the 12^{th} egg if the average weight of the last two eggs did not reach or exceed 47.3 g (industry standard “settable” egg weight). Once the first egg was laid by a given hen, her eggs were weighed for no longer than 4 weeks. To assess the effects of vaccine treatments on egg size of hens at maturity, each hen's daily egg weight (g) was also recorded during a 4-day period of lay beginning at 32 wk of age. Eggs were marked on their shell surfaces to indicate the hen's treatment group color and wing badge number as well as the hen's pen number. This allowed association of a given hen's egg with her specific injection treatment by reference to the study blind wing badge assignments.

Body Weight Body weight (BWT) was measured weekly from 13 to 26 weeks of age according to procedures in Example 2.

Mortality and Necropsy Daily mortality MORT records were maintained and all birds that expired during the study were examined for abnormalities, pathologies and probable cause of death. Mortalities were recorded as KT (killed terminal, no necropsy required), KM (killed morbid, necropsy required), D (dead, necropsy required) or RM (removed mechanical, no necropsy required).

Anti-inhibin Antibody Titers Relative titers of antibodies directed against the ∝-subunit of inhibin were measured by an ELISA (Viro Dynamics) in blood samples collected at 18 weeks, 4 days of age and 20 weeks of age; these blood samplings corresponded to 4 days and 2 weeks post-booster injections, respectively.

Statistical Analyses Indices of puberty (PS, FIRST, and TFIVE), HDEP (weekly and cumulative), EWT, MORT, and titer ELISA data were analyzed by procedures similar to those described by Satterlee et al. (2002) Poultry Sci. 81: 519-528.

Results and Discussion The low dose of ¹⁻²⁶INH-MAP (0.05 mg) significantly enhanced (primarily P<0.05) PS beginning at 21 wk of age and persisted until 24 weeks of age. The high dose (0.1 mg of ¹⁻²⁶INH-MAP) increased PS at these times but this increase did not attain statistical significance.

Because the flock was photostimulated at 20 weeks, 5 days of age, the finding of the first statistical difference in PS between the controls and inhibin-immunized pullets by only two days later (at 21 weeks of age) indicates that vaccination against inhibin may accelerate puberty somewhat independent of the effects of lighting and its well known action of bringing about elevations in FSH needed to mature the ovary (via FSH-induced acceleration of follicular recruitment and development in anticipation of egg lay).

Significant elevations in post-peak HDEP laying rates were observed for certain weeks and dosages within ¹⁻²⁶INH-MAP-immunized hens when compared to control responses. Specifically, these differences all occurred during the second half of the trial (i.e., during 9 to 16 wk of lay) an indication that ¹⁻²⁶INH-MAP was prolonging the persistency of egg lay. Peak HDEP is known to occur at about 8 wk of lay in Cobb broiler breeders as was the case for the controls in the present study. The post-peak HDEP differences between the controls and hens treated with the low dose of ¹⁻²⁶INH-MAP were dramatic-HDEP elevations (in each case about 10%) were seen at 9 (P<0.10), 13 (P<0.05), and 16 (P<0.05) wk of lay.

In support of the weekly HDEP data presented earlier, when compared to the control response, both doses of ¹⁻²⁶INH-MAP showed reductions (3.7 and 2-fold, respectively, for the low and high dose) in the numbers of hens that did not lay an egg during the last two weeks of the trial. The low dose of ¹⁻²⁶INH-MAP response was a statistically relevant reduction (P<0.03) when compared to the controls. These data demonstrate a significant reduction in the numbers of hens vaccinated against inhibin that do not lay which results in direct economic gain for the producer.

Effects of active immunization against inhibin in broiler breeders on egg lay were also examined in hens having body weights that were below the target body weight at lighting as opposed to heavier hens who had reached or exceeded their target BWT. Specifically, the effects of the vaccine on hen-housed egg production (HHEP) were tested in “light” vs. “heavy” hen subpopulations using data from the full 16 weeks of lay and using data only from the last 8 weeks of lay (9 to 16 weeks of lay). For simplicity and in order to insure sufficient numbers of observations (hens) in the categorical BWT treatment groups to permit meaningful statistical tests, data from the hens of the low and high doses within each test article were combined.

The target body weight for Cobb-500 breeder hens at 21 weeks of age (at lighting) is 2320 g. The flock average BWT at this time was 2132 g or approximately 92% of target. Therefore, within each injection treatment, hens having a BWT at 21 weeks of ≧2132 g were designated as “heavy” hens and the residual hens of BWT <2132 g were considered to be “light” hens. This classification system resulted in mean BWTs of light and heavy hen treatments that were 1947 and 2305 g, respectively (or, approximately 84 and 99 % of the target BWT, respectively).

Light BWT hens receiving ¹⁻²⁶INH-MAP statistically (generally P<0.05) outperformed the light BWT controls (i.e., ¹⁻²⁶INH-MAP-immunized birds had higher HHEP when either data from the entire study, full 16 weeks of lay (P<0.08), or from only the second half of lay, 9 to 16 weeks of lay (P<0.05), were considered). These increases in HHEP over control responses were marked (about14%) for each of the two periods of study. In fact, immunizing light hens against inhibin resulted in an elevated HHEP that was markedly similar to the elevated HHEP observed in heavy hens regardless of their injection treatment. On the other hand, no differences in HHEP were detected between the heavy controls and either of the two inhibin-immunized heavy hen treatment groups (full 16 weeks of lay or 9 to 16 weeks of lay). These findings suggest that producers could gain substantial economic benefit by purposely feeding birds to a lighter BWT at photostimulation.

Mean relative titers of antibodies directed against the ∝-subunit of chicken inhibin, as measured by a commercially developed ELISA, in blood samples collected at 18 weeks, 4 days of age (4 days after booster injections) and 20 weeks of age (2 weeks after booster injections) showed that inhibin-based antigens (regardless of dose) exhibited robust (P<0.01) titer ELISA responses when compared to control responses.

To assess the effects of vaccine treatments on early egg size, the first 12 eggs laid (trap-nested) by each hen of each injection treatment were individually weighed. Vaccine treatments did not affect EWT of the first 12 eggs (data not shown) nor did they affect the age at which hens reached the industry standard “settable” egg weight of 47.3 g (data not shown). These findings indicate no ill effects of vaccine administration on egg size, a variable that can greatly impact egg hatchability. Also, injection treatments did not alter egg size at maturity (peak egg production; 32 weeks of age) as evidenced by no differences in EWTs among treatments (data not shown). Weekly BWTs (15 to 26 weeks of age) and trial-wise MORT (from time of primary challenges to the end of the trial, 16 weeks of lay) were also unaffected by treatment with the ¹⁻²⁶INH-MAP vaccine (data not shown).

### EXAMPLE 4

### Enhancing Production Performance In Quail

The ¹⁻²⁶INH-MAP composition of the present invention is used as an antigen to immunize prepubescent, female Japanese quail against circulating inhibin levels, and to therefore accelerate the onset of egg lay in the treated quail. The method described in Example 2 is followed with the following exceptions. The average age at puberty for an untreated quail is approximately six to eight weeks. The following is the treatment schedule for Japanese quail having an approximate body weight range of 0.1 to 0.25 pounds: primary (first) injection of between about 0.025 to 0.1 mg of the ¹⁻²⁶INH-MAP composition of the present invention on its 25th day of age; and a booster of 0.01 to 0.05 mg between 32 to 35 days of age.

### EXAMPLE 5

### Enhancing Production Performance In Turkeys

The ¹⁻²⁶INH-MAP composition of the present invention is used as an antigen to immunize prepubescent, female turkeys against circulating inhibin levels, and to therefore accelerate the onset of egg lay in the treated turkeys. The method described in Example 2 is followed with the following exceptions. The average age at puberty for an untreated turkey is approximately 30 weeks. The following is a treatment schedule for a turkey having an approximate body weight range of 9.0 to 12 pounds: primary (first) injection of about 0.1 to about 0.5 mg of the ¹⁻²⁶INH-MAP composition of the present invention on its 24th week of age and a booster of between about 0.05 to about 0.25 mg on the 27th week of age.

All patents, publications and abstracts cited above are incorporated herein by reference in their entirety. It should be understood, of course, that the foregoing relates only to preferred embodiments of the present invention and that numerous modifications or alterations may be made therein without departing from the spirit and the scope of the invention as set forth in the appended claims.

1. A composition comprising a multiple antigenic peptide comprising at least two inhibin-related peptides linked to a multilinker backbone.

2. The composition of claim 1, comprising a multiple antigenic peptide described by formula I
B-(L-P)n I
wherein B is a multilinker backbone, n is an integer from 2 to about 20, each L is a covalent bond or a linking group which may be present or absent, and each P is an inhibin-related peptide, or conservative substitution thereof, having from about 4 to about 115 amino acid residues.

3. The composition of claim 2, wherein B is 3, L is absent, n is 4 and the composition is described by formula VII

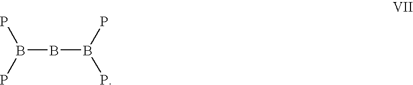

4. The composition of claim 3, wherein at least one P is SEQ ID No: 1 or a conservative substitution thereof.

5. The composition of claim 1, wherein the composition is described by any one of the following formulae:
P—(B—P)m-B—P II
wherein m is an integer from zero to about twenty;
Pa—(B)n-Pa III
wherein n is an integer from 1 to 20, and a is 1 or 2; 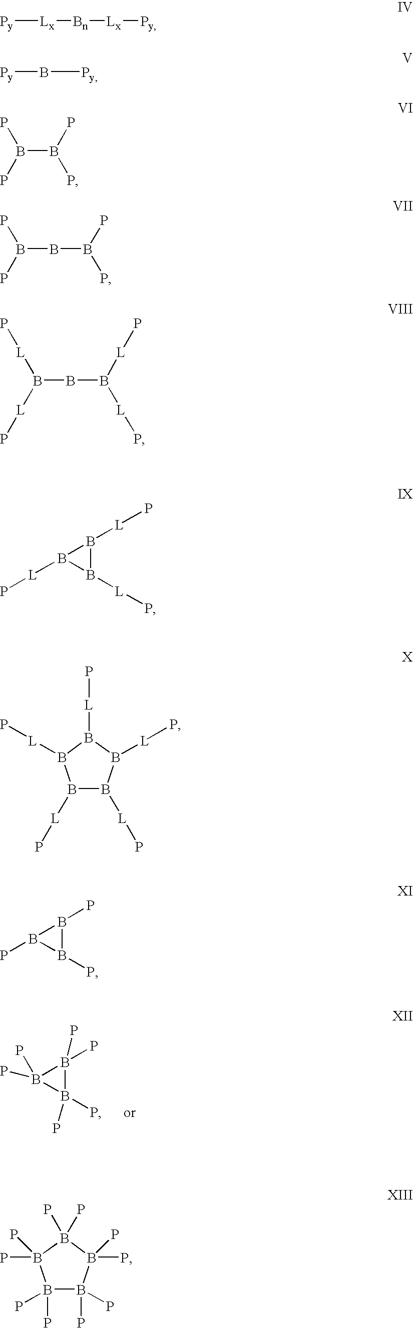
wherein P is an inhibin-related peptide, or a conservative substitution thereof, having from about 4 to about 115 amino acid residues, B is a multilinker backbone, n is an integer from 1 to 20, each L is a covalent bond or a linking group which may be present or absent, y is 1 or 2, x is an integer from 1 to 3.

6. The composition of claim 4 described by formula XIV

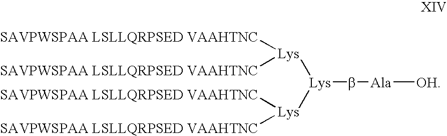

7. The composition of claim 4 described by formula XV

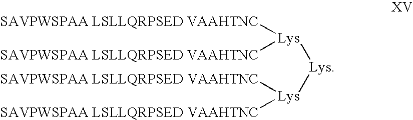

8. A method of enhancing production performance comprising administration of an effective amount of a composition comprising a multiple antigenic peptide comprising at least two inhibin-related peptides linked to a backbone and an acceptable carrier to an animal, wherein the amount is effective to enhance production performance in the animal.

9. The method of claim 8, wherein the composition comprises a multiple antigenic peptide described by formula I
B-(L-P)n I
wherein B is a multilinker backbone, n is an integer from 2 to about 20, each L is a covalent bond or a linking group which may be present or absent, and each P is an inhibin-related peptide, or a conservative substitution thereof, having from about 4 to about 115 amino acid residues.

10. The method of claim 9, wherein B is 3, L is absent, n is 4 and the composition is described by formula VII

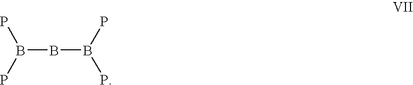

11. The method of claim 8, wherein the composition is described by any one of the following formulae:
P—(B—P)m-B—P II
wherein m is an integer from zero to about twenty;
Pa—(B)n-Pa III
wherein n is an integer from 1 to 20, and a is 1 or 2; 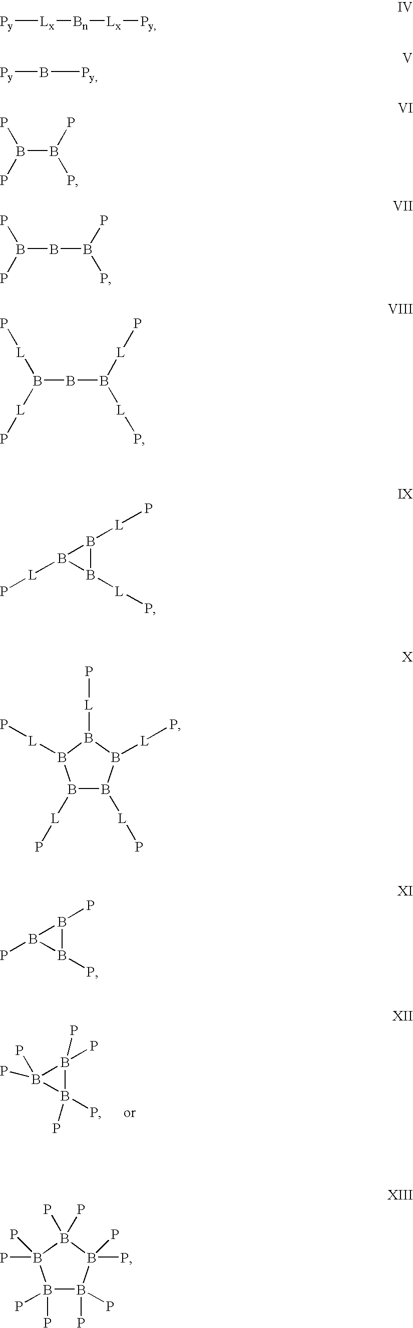
wherein P is an inhibin-related peptide, or a conservative substitution thereof, having from about 4 to about 115 amino acid residues, B is a multilinker backbone, n is an integer from 1 to 20, each L is a covalent bond or a linking group which may be present or absent, y is 1 or 2, x is an integer from 1 to 3.

12. The method of claim 10, wherein the composition is described by formula XIV

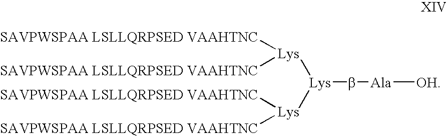

13. The method of claim 10, wherein the compositions is described by formula XV.

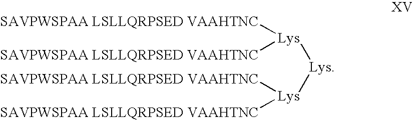

14. The method of claim 8, wherein enhancing production performance comprises increasing egg lay in animals.

15. The method of claim 8, wherein the animal is a bird.

16. The method of claim 15, wherein the bird is a chicken, turkey, quail, goose or duck.

17.-20. (canceled)

21. The method of claim 9, wherein enhancing production performance comprises increasing egg lay in animals.

22. The method of claim 10, wherein enhancing production performance comprises increasing egg lay in animals.

23. The method of claim 11, wherein enhancing production performance comprises increasing egg lay in animals.

24. The method of claim 12, wherein enhancing production performance comprises increasing egg lay in animals.

25. The method of claim 13, wherein enhancing production performance comprises increasing egg lay in animals.
